(19) United States
(12) Patent Application Publication
(10) Pub. No.: US 2016/0102301 A1
(43) Pub. Date: Apr. 14, 2016

(54) PROTEIN, METHOD FOR MANUFACTURING SAME, AND METHOD FOR EVALUATING PROTEIN ACTIVITY

(71) Applicant: UBE INDUSTRIES, LTD., Ube-shi, Yamaguchi (JP)

(72) Inventors: Katsuki SUZUKI, Ube-shi, Yamaguchi (JP); Youichi YOSHIDA, Ube-shi, Yamaguchi (JP)

(21) Appl. No.: 14/895,356

(22) PCT Filed: Aug. 7, 2014
(86) PCT No.: PCT/JP2014/070917

§ 371 Date: Dec. 2, 2015

(30) Foreign Application Priority Data
Aug. 9, 2013 (JP) 2013-166686
Mar. 17, 2014 (JP) 2014-053718

(51) Int. Cl. C12N 11/08; G01N 21/35; C12N 9/08; C12N 9/20; C07K 16/00; C07K 1/00

(52) U.S. Cl. CPC C12N 11/08; C07K 16/00; C07K 1/00; C12N 9/0065; C12N 9/20; G01N 21/35

(57) ABSTRACT

The present invention relates to a method for producing a protein, comprising an inspection process, wherein the inspection process comprises: a step of approximating an infrared absorption band derived from a protein appearing around 1500 to 1600 cm⁻¹ or around 1600 to 1700 cm⁻¹ in an infrared absorption spectrum of the protein, by one or more normal distributions, a step of calculating an index value indicating a degree of broadening of the infrared absorption band based on the normal distributions, and a step of comparing the index value with a predetermined threshold to select, as a good-quality product, a protein having a degree of broadening of the infrared absorption band that is smaller than the threshold.

### TECHNICAL FIELD

The present invention relates to a protein, a method for producing the same, and a method for evaluating the activity of the protein. The present invention also relates to an immobilized lipase and a reactivated immobilized lipase, a method for producing them, and a method for evaluating the activity of the lipase. The present invention further relates to an immobilized peroxidase, a method for producing the same, and a method for evaluating the activity of the peroxidase. The present invention still further relates to an antibody, a method for producing the same, and a method for evaluating the activity of the antibody.

### BACKGROUND ART

Conventionally, the activity of a protein has been evaluated, for example, by actually carrying out a catalytic reaction or the like and then measuring the activity thereof.

For example, an immobilized lipase (e.g., see Patent Literature 1), which has been industrially widely used in reactions including esterification reactions of various types of carboxylic acids such as fatty acid with alcohols such as monoalcohol and polyhydric alcohol, transesterification reactions between a plurality of carboxylic acid esters, and the like, may lose a sufficient catalytic activity due to conditions for preparation thereof, the use thereof in such reactions, etc.; however, the catalytic activity of such an immobilized lipase has been evaluated by actually carrying out a transesterification reaction or the like, and then measuring the activity thereof.

Meanwhile, an example of analyzing the higher-order structure of a lipase in such an immobilized lipase has been known. For example, Non Patent Literature 1 describes methods for analyzing the structure of a lipase immobilized on a solid particle according to a circular dichroism (CD) method, a diffuse reflectance infrared Fourier transform (DRIFT) spectrometry, and a tryptophan residue fluorescence method. Non Patent Literature 2 describes that the influence of raw materials, the presence or absence of a water content, and the contact with biodiesel upon the catalytic, enzymatic and physical safety of Novozym (registered trademark) 435 in biodiesel production of using enzyme has been analyzed. Non Patent Literature 3 describes that the influence of a pretreatment with an organic solvent on the initial activity and secondary structure of an immobilized lipase derived from Pseudomonas (Pseudomonas cepacia) has been analyzed.

### CITATION LIST

### Patent Literature

Patent Literature 1: Japanese Patent Application Laid-Open No. 2011-207975

### Non Patent Literature

Non Patent Literature 1: ChemPhysChem, Vol. 10, pp. 1492-1499 (2009)

Non Patent Literature 2: Catalysis Today, Vol. 213, pp. 73-80 (2013)

Non Patent Literature 3: Process Biochemistry, Vol. 45, pp. 1176-1180 (2010)

### SUMMARY OF INVENTION

### Problems to be Solved by the Invention

At present, a protein has been widely used as a catalyst in industrial processes or as a pharmaceutical agent. If the activity of a protein is reduced, it leads to a reduction in the efficiency of the entire process, a reduction in therapeutic effects, and the like; and thus, it is extremely important to evaluate the activity of a protein and to provide a protein having a sufficient activity.

Moreover, with regard to a protein having an insufficient activity, there are two cases, namely, a case in which the activity of a protein can be reactivated, for example, by adjusting the water content percentage of the protein (hereinafter also referred to as “reversible denaturation”), and a case in which the activity of a protein cannot be reactivated (hereinafter referred to as “irreversible denaturation”).

The conventional evaluation of the activity of a protein has been carried out by actually performing a catalytic reaction or the like, and then measuring the activity thereof. In such an evaluation method, when the activity of a protein is not sufficient, it is difficult to evaluate whether it is caused by reversible denaturation or irreversible denaturation.

Furthermore, there have not yet been sufficient options regarding methods capable of evaluating the activity of a protein without actually carrying out a catalytic reaction or the like. For example, in the case of an industrially applicable immobilized lipase, Non Patent Literatures 1 to 3 describe the high-relationship between the structure of a lipase in an immobilized lipase and the catalytic activity. However, Non Patent Literature 1 describes that, in the case of Novozym (registered trademark) 435 (manufactured by Novozymes) formed by immobilization of lipase CalB on a porous resin carrier, since strong absorption derived from the porous resin carrier is adjacent to absorption derived from the lipase, structural analysis cannot be carried out by measuring an infrared absorption spectrum.

Further, Non Patent Literature 2 describes that α-helix has decreased and β-sheet has increased in a lipase whose activity has been reduced due to the use thereof in reactions, although they are not quantitative data. Non Patent Literature 3 describes that the structures of α-helix and β-sheet have been changed by being treated with an organic solvent, but contrary to Non Patent Literature 2, Non Patent Literature 3 describes that the activity has been improved together with such structural changes.

Hence, under the current circumstances, opposite reports have been made regarding the correlation between the structural change of a lipase and a catalytic activity thereof, and whether or not catalytic ability can be evaluated based on the measurement of an infrared absorption spectrum has not been clarified. Moreover, none of Non Patent Literatures 1 to 3 contains any description or suggestion regarding a technical means for distinguishing reversible denaturation from irreversible denaturation.

The present invention has been made under such circumstances, and it is an object of the present invention to provide a method for producing a protein, by which a protein having a sufficient activity can be obtained, and a protein obtained by this production method. It is another object of the present invention to provide a method for evaluating the activity of a protein, by which the activity of a protein can be evaluated without actually measuring the activity.

### Means for Solving the Problems

The present inventors have found that an index value indicating a degree of broadening of an infrared absorption band, which is obtained by approximating an infrared absorption band with a specific wavenumber of a protein in the infrared absorption spectrum of the protein by a normal distribution or normal distributions and then calculating it based on the normal distribution(s), reflects an activity or a potential activity of the protein. That is to say, the inventors have found that a protein having a sufficient activity can be obtained by selecting a protein based on the aforementioned index value. The present invention is based on this novel finding.

Specifically, the present invention relates to a method for producing a protein, comprising an inspection process, wherein the inspection process comprises: a step of approximating an infrared absorption band derived from a protein appearing around 1500 to 1600 cm⁻¹ (hereinafter also referred to as an “absorption band II”) or an infrared absorption band derived from a protein appearing around 1600 to 1700 cm⁻¹ (hereinafter also referred to as an “absorption band I”) in the infrared absorption spectrum of the protein by one or more normal distributions, a step of calculating an index value indicating a degree of broadening of the infrared absorption band based on the normal distributions, and a step of comparing the index value with a predetermined threshold to select, as a good-quality product, a protein having a degree of broadening of the infrared absorption band that is smaller than the threshold.

According to the method for producing a protein of the present invention, since the production method has the above described inspection process, a protein having an activity can be produced. In addition, since the reversible denaturation of a protein activity can be distinguished from the irreversible denaturation thereof, a protein capable of reactivating an activity can be obtained.

The above described index value may be a half-value width of a single normal distribution, when the infrared absorption band is approximated by the single normal distribution. Since the half-value width correlates with the degree of broadening of the infrared absorption band, it is possible to select a protein having an activity or a protein capable of reactivating an activity, by using the half-value width as an index value.

The above described index value may be a value obtained by subjecting the infrared absorption band to waveform separation to obtain multiple normal distributions, and then dividing a sum of the areas of one or more normal distributions around the peak top position of the infrared absorption band by a sum of the areas of one or more normal distributions around the end of the infrared absorption band (hereinafter also referred to as an “absorption band area ratio”). Since the absorption band area ratio correlates with the degree of broadening of the infrared absorption band, it is possible to select a protein having an activity or a protein capable of reactivating an activity, by using the absorption band area ratio as an index value.

The absorption band area ratio may be a value which is obtained by subjecting the infrared absorption band to waveform separation to obtain two normal distributions each having a peak around the peak top position of the infrared absorption band and having a different half-value width, and then dividing the area of a normal distribution having a smaller half-value width among the two normal distributions by the area of a normal distribution having a larger half-value width.

The absorption band area ratio may be a value obtained by subjecting the infrared absorption band to waveform separation to obtain an n number of normal distributions A₁ to Aₙ (wherein n is an integer of 3 or greater), and either when the number n is an even number, by dividing a sum of the area(s) of at least one or both of A_{n/2} and A_{n/2+1} by a sum of the area of at least one selected from the group consisting of A₁ to A_{n/2-1} and A_{n/2+2} to Aₙ, or when the number n is an odd number, by dividing the area of A_{(n+1)/2} by a sum of the area of at least one selected from the group consisting of A₁ to A_{(n+1)/2-1} and A_{(n-1)/2+2} to Aₙ.

The above described infrared absorption spectrum is preferably measured by an attenuated total reflection method. By this, it is possible to select a protein more precisely, by using the above described index value.

The present invention also provides a protein obtained by the above described method for producing a protein. Since the protein of the present invention is obtained by the above described production method, it has an activity or is capable of reactivating a sufficient activity.

The present invention also relates to a method for producing an immobilized lipase formed by immobilizing a lipase on a resin carrier, wherein the production method has an inspection process comprising: a step of approximating an infrared absorption band derived from a lipase appearing around 1500 to 1600 cm⁻¹ (absorption band II) or an infrared absorption band derived from a lipase appearing around 1600 to 1700 cm⁻¹ (absorption band I) in the infrared absorption spectrum of the immobilized lipase by one or more normal distributions, a step of calculating an index value indicating a degree of broadening of the infrared absorption band based on the normal distributions, and a step of comparing the index value with a predetermined threshold to select, as a good-quality product, an immobilized lipase having a degree of broadening of the infrared absorption band that is smaller than the threshold.

According to the method for producing an immobilized lipase of the present invention, since this method has a step of comparing the calculated index value with a threshold and selecting an immobilized lipase having a degree of broadening of the infrared absorption band that is smaller than the threshold, an immobilized lipase that has a catalytic activity or is capable of reactivating a catalytic activity can be obtained.

In the method for producing an immobilized lipase of the present invention, an immobilized lipase, in which the index value is a half-value width of a single normal distribution, when absorption band I is approximated by the single normal distribution (hereinafter also referred to as an “index value 1”), and in the selection step, the index value becomes 70 cm⁻¹ or less, may be selected as a good-quality product.

In the method for producing an immobilized lipase of the present invention, an immobilized lipase, in which the index value is a value obtained by subjecting absorption band I to waveform separation to obtain two normal distributions A₁ (peak position: 1656 cm⁻¹, half-value width: 47 cm⁻¹) and A₂ (peak position: 1656 cm ⁻¹, half-value width: 82 cm ⁻¹), so that an absolute value of a difference between the area of absorption band I and a sum of the areas of the two normal distributions becomes a minimum, and then dividing the area of A₁ by the area of A₂ (hereinafter also referred to as an “index value 2”), and in the selection step, the index value becomes 0.27 or more, may be selected as a good-quality product.

In the method for producing an immobilized lipase of the present invention, an immobilized lipase, in which the index value is a value obtained by subjecting absorption band I to waveform separation to obtain three normal distributions A₁ (peak position: 1680 cm⁻¹, half-value width: 50 cm⁻¹), A₂ (peak position: 1656 cm⁻¹, half-value width: 50 cm⁻¹) and A₃ (peak position: 1631 cm⁻¹, half-value width: 50 cm⁻¹), so that an absolute value of a difference between the area of absorption band I and a sum of the areas of the three normal distributions becomes a minimum, and then dividing the area of A₂ by a sum of the areas of A₁ and A₃ (hereinafter also referred to as an “index value 3”), and in the selection step, the index value becomes 0.9 or more, may be selected as a good-quality product.

In the method for producing an immobilized lipase of the present invention, an immobilized lipase, in which the index value is a value obtained by subjecting absorption band I to waveform separation to obtain five normal distributions A₁ (peak position: 1685 cm⁻¹, half-value width: 30 cm⁻¹), A₂ (peak position: 1670 cm⁻¹, half-value width: 30 cm⁻¹), A₃ (peak position: 1656 cm⁻¹, half-value width: 30 cm⁻¹), A₄ (peak position: 1641 cm⁻¹, half-value width: 30 cm⁻¹) and A₅ (peak position: 1626 cm⁻¹, half-value width: 30 cm⁻¹), so that an absolute value of a difference between the area of absorption band I and a sum of the areas of the five normal distributions becomes a minimum, and then dividing the area of A₃ by a sum of the areas of A₁, A₂, A₄ and A₅ (hereinafter also referred to as an “index value 4”), and in the selection step, the index value becomes 0.35 or more, may be selected as a good-quality product.

In the method for producing an immobilized lipase of the present invention, an immobilized lipase, in which the index value is a value obtained by subjecting absorption band I to waveform separation to obtain eight normal distributions A₁ (peak position: 1692 cm⁻¹, half-value width: 19 cm⁻¹), A₂ (peak position: 1682 cm⁻¹, half-value width: 19 cm⁻¹), A₃ (peak position: 1670 cm⁻¹, half-value width: 19 cm⁻¹), A₄ (peak position: 1658 cm⁻¹, half-value width: 19 cm⁻¹), A₅ (peak position: 1648 cm⁻¹, half-value width: 19 cm⁻¹), A₆ (peak position: 1638 cm⁻¹, half-value width: 19 cm⁻¹), A₇ (peak position: 1629 cm⁻¹, half-value width: 19 cm⁻¹) and A₈ (peak position: 1619 cm⁻¹, half-value width: 19 cm⁻¹), so that an absolute value of a difference between the area of absorption band I and a sum of the areas of the eight normal distributions becomes a minimum, and then dividing a sum of the areas of A₄ and A₅ by a sum of the areas of A₁, A₂, A₃, A₆, A₇ and A₈ (hereinafter also referred to as an “index value 5”), and in the selection step, the index value becomes 0.6 or more, may be selected as a good-quality product.

In the method for producing an immobilized lipase of the present invention, an immobilized lipase, in which the index value is a value obtained by subjecting absorption band I to waveform separation to obtain eight normal distributions A₁ (peak position: 1692 cm⁻¹, half-value width: 19 cm⁻¹), A₂ (peak position: 1682 cm⁻¹, half-value width: 19 cm⁻¹), A₃ (peak position: 1670 cm⁻¹, half-value width: 19 cm⁻¹), A₄ (peak position: 1658 cm⁻¹, half-value width: 19 cm⁻¹), A₅ (peak position: 1648 cm⁻¹, half-value width: 19 cm⁻¹), A₆ (peak position: 1638 cm⁻¹, half-value width: 19 cm⁻¹), A₇ (peak position: 1629 cm⁻¹, half-value width: 19 cm⁻¹) and A₈ (peak position: 1619 cm⁻¹, half-value width: 19 cm⁻¹), so that an absolute value of a difference between the area of absorption band I and a sum of the areas of the eight normal distributions becomes a minimum, and then dividing a sum of the areas of A₄ and A₅ by a sum of the areas of A₂, A₃ and A₈ (hereinafter also referred to as an “index value 6”), and in the selection step, the index value becomes 1.2 or more, may be selected as a good-quality product.

In the method for producing an immobilized lipase of the present invention, an immobilized lipase, in which the index value is a half-value width of a single normal distribution, when absorption band II is approximated by the single normal distribution (hereinafter also referred to as an “index value 7”), and in the selection step, the index value becomes 44 cm⁻¹ or less, may be selected as a good-quality product.

In the method for producing an immobilized lipase of the present invention, an immobilized lipase, in which the index value is a value obtained by subjecting absorption band II to waveform separation to obtain three normal distributions B₁ (peak position: 1570 cm⁻¹, half-value width: 31 cm⁻¹), B₂ (peak position: 1545 cm⁻¹, half-value width: 31 cm⁻¹) and B₃ (peak position: 1518 cm⁻¹, half-value width: 31 cm⁻¹), so that an absolute value of a difference between the area of absorption band II and a sum of the areas of the three normal distributions becomes a minimum, and then dividing the area of B₂ by a sum of the areas of B₁ and B₃ (hereinafter also referred to as an “index value 8”), and in the selection step, the index value becomes 1.2 or more, may be selected as a good-quality product.

The above described immobilized lipase may have a transesterification activity or an ester hydrolysis activity.

The above described infrared absorption spectrum is preferably measured by an attenuated total reflection method. By this, it is possible to select an immobilized lipase more precisely, by using the above described index value.

The lipase may be a lipase derived from Burkholderia cepacia or Candida antarctica.

The present invention also provides an immobilized lipase obtained by the above described method for producing an immobilized lipase. Since the immobilized lipase of the present invention is obtained by the above described production method, it has a catalytic activity, or is capable of reactivating a sufficient catalytic activity.

The present invention also relates to a method for producing an immobilized peroxidase formed by immobilizing a peroxidase on a silica carrier, wherein the production method has an inspection process comprising: a step of approximating an infrared absorption band derived from a peroxidase appearing around 1500 to 1600 cm⁻¹ (absorption band II) or an infrared absorption band derived from a peroxidase appearing around 1600 to 1700 cm⁻¹ (absorption band I) in the infrared absorption spectrum of the immobilized peroxidase by one or more normal distributions, a step of calculating an index value indicating a degree of broadening of the infrared absorption band based on the normal distributions, and a step of comparing the index value with a predetermined threshold to select, as a good-quality product, an immobilized peroxidase having a degree of broadening of the infrared absorption band that is smaller than the threshold.

According to the method for producing an immobilized peroxidase of the present invention, since this method has a step of comparing the calculated index value with a threshold and selecting an immobilized peroxidase having a degree of broadening of the infrared absorption band that is smaller than the threshold, an immobilized lipase that has a catalytic activity or is capable of reactivating a catalytic activity can be obtained.

In the method for producing an immobilized peroxidase of the present invention, an immobilized peroxidase, in which the index value is index value 7, and in the selection step, the index value becomes 75 cm⁻¹ or less, may be selected as a good-quality product.

In the method for producing an immobilized peroxidase of the present invention, an immobilized peroxidase, in which the index value is index value 8, and in the selection step, the index value becomes 0.45 or more, may be selected as a good-quality product.

The above described infrared absorption spectrum is preferably measured by an attenuated total reflection method. By this, it is possible to select an immobilized peroxidase more precisely, by using the above described index value.

The present invention also provides an immobilized peroxidase obtained by the above described method for producing an immobilized peroxidase. Since the immobilized peroxidase of the present invention is obtained by the above described production method, it has a catalytic activity, or is capable of reactivating a sufficient catalytic activity.

The present invention also relates to a method for producing an antibody, wherein the production method has an inspection process comprising: a step of approximating an infrared absorption band derived from an antibody appearing around 1500 to 1600 cm⁻¹ (absorption band II) or an infrared absorption band derived from an antibody appearing around 1600 to 1700 cm⁻¹ (absorption band I) in the infrared absorption spectrum of the antibody by one or more normal distributions, a step of calculating an index value indicating a degree of broadening of the infrared absorption band based on the normal distributions, and a step of comparing the index value with a predetermined threshold to select, as a good-quality product, an antibody having a degree of broadening of the infrared absorption band that is smaller than the threshold.

According to the method for producing an antibody of the present invention, since this method has a step of comparing the calculated index value with a threshold and selecting an antibody having a degree of broadening of the infrared absorption band that is smaller than the threshold, an antibody having a sufficient titer can be obtained.

In the method for producing an antibody of the present invention, an antibody, in which the index value is index value 1, and in the selection step, the index value becomes 65 cm⁻¹ or less, may be selected as a good-quality product.

In the method for producing an antibody of the present invention, an antibody, in which the index value is index value 6, and in the selection step, the index value becomes 0.98 or more, may be selected as a good-quality product.

In the method for producing an antibody of the present invention, an antibody, in which the index value is index value 8, and in the selection step, the index value becomes 0.85 or more, may be selected as a good-quality product.

The above described infrared absorption spectrum is preferably measured by an attenuated total reflection method. By this, it is possible to select an antibody more precisely, by using the above described index value.

The present invention also provides an antibody obtained by the above described method for producing an antibody. Since the antibody of the present invention is obtained by the above described production method, it has a sufficient titer.

The present invention also relates to a method for producing a reactivated immobilized lipase, in which a lipase activity is partially or totally reactivated, from an immobilized lipase having a reduced lipase activity, wherein the immobilized lipase is formed by immobilizing a lipase on a resin carrier, and wherein the method comprises a selection process, the selection process comprising: a step of approximating an infrared absorption band derived from a lipase appearing around 1500 to 1600 cm⁻¹ (absorption band II) or an infrared absorption band derived from a lipase appearing around 1600 to 1700 cm⁻¹ (absorption band I) in the infrared absorption spectrum of the immobilized lipase having a reduced lipase activity by one or more normal distributions, a step of calculating an index value indicating a degree of broadening of the infrared absorption band based on the normal distributions, and a step of comparing the index value with a predetermined threshold to select, as an immobilized lipase in which a lipase activity is possibly reactivated, the immobilized lipase having a reduced lipase activity that has a degree of broadening of the infrared absorption band that is smaller than the threshold.

According to the method for producing a reactivated immobilized lipase of the present invention, since this method has a step of comparing the calculated index value with a threshold and selecting an immobilized lipase having a degree of broadening of the infrared absorption band that is smaller than the threshold, a reversibly denaturable immobilized lipase can be selected from among immobilized lipases having a reduced lipase activity. That is to say, an immobilized lipase capable of reactivating a sufficient catalytic activity can be obtained.

In the method for producing a reactivated immobilized lipase of the present invention, an immobilized lipase, in which the index value is index value 1, and in the selection step, the index value becomes 70 cm⁻¹ or less, may be selected as an immobilized lipase in which a lipase activity is possibly reactivated.

In the method for producing a reactivated immobilized lipase of the present invention, an immobilized lipase, in which the index value is index value 2, and in the selection step, the index value becomes 0.27 or more, may be selected as an immobilized lipase in which a lipase activity is possibly reactivated.

In the method for producing a reactivated immobilized lipase of the present invention, an immobilized lipase, in which the index value is index value 3, and in the selection step, the index value becomes 0.9 or more, may be selected as an immobilized lipase in which a lipase activity is possibly reactivated.

In the method for producing a reactivated immobilized lipase of the present invention, an immobilized lipase, in which the index value is index value 4, and in the selection step, the index value becomes 0.35 or more, may be selected as an immobilized lipase in which a lipase activity is possibly reactivated.

In the method for producing a reactivated immobilized lipase of the present invention, an immobilized lipase, in which the index value is index value 5, and in the selection step, the index value becomes 0.6 or more, may be selected as an immobilized lipase in which a lipase activity is possibly reactivated.

In the method for producing a reactivated immobilized lipase of the present invention, an immobilized lipase, in which the index value is index value 6, and in the selection step, the index value becomes 1.2 or more, may be selected as an immobilized lipase in which a lipase activity is possibly reactivated.

In the method for producing a reactivated immobilized lipase of the present invention, an immobilized lipase, in which the index value is index value 7, and in the selection step, the index value becomes 44 cm⁻¹ or less, may be selected as an immobilized lipase in which a lipase activity is possibly reactivated.

In the method for producing a reactivated immobilized lipase of the present invention, an immobilized lipase, in which the index value is index value 8, and in the selection step, the index value becomes 1.2 or more, may be selected as an immobilized lipase in which a lipase activity is possibly reactivated.

The above described method for producing a reactivated immobilized lipase may comprise a reactivation process for treating the immobilized lipase selected in the selection process with water or a hydrous organic solvent. The immobilized lipase selected in the selection process is capable of reactivating a lipase activity by undergoing the above described reactivation process.

The above described immobilized lipase may have a transesterification activity or an ester hydrolysis activity.

The above described infrared absorption spectrum is preferably measured by an attenuated total reflection method. By this, it is possible to select an immobilized lipase more precisely, by using the above described index value.

The lipase may be a lipase derived from Burkholderia cepacia or Candida antarctica.

The present invention also provides a reactivated immobilized lipase obtained by the above described method for producing a reactivated immobilized lipase. Since the reactivated immobilized lipase of the present invention is obtained by the above described production method, it has a sufficient catalytic activity, or is capable of reactivating a sufficient catalytic activity.

The present invention can be also considered to be a method for evaluating the activity of a protein, comprising an evaluation process, wherein the evaluation process comprises: a step of approximating an infrared absorption band derived from a protein appearing around 1500 to 1600 cm⁻¹ (absorption band II) or an infrared absorption band derived from a protein appearing around 1600 to 1700 cm⁻¹ (absorption band I) in the infrared absorption spectrum of the protein by one or more normal distributions, a step of calculating an index value indicating a degree of broadening of the infrared absorption band based on the normal distributions, and a step of comparing the index value with a predetermined threshold to evaluate, as a protein having an activity, a protein having a degree of broadening of the infrared absorption band that is smaller than the threshold.

The present invention can be also considered to be a method for evaluating the lipase activity of an immobilized lipase formed by immobilizing a lipase on a resin carrier, wherein the evaluation method has an evaluation process comprising: a step of approximating an infrared absorption band derived from a lipase appearing around 1500 to 1600 cm⁻¹ (absorption band II) or an infrared absorption band derived from a lipase appearing around 1600 to 1700 cm⁻¹ (absorption band I) in the infrared absorption spectrum of the immobilized lipase by one or more normal distributions, a step of calculating an index value indicating a degree of broadening of the infrared absorption band based on the normal distributions, and a step of comparing the index value with a predetermined threshold, and evaluating an immobilized lipase having a degree of broadening of the infrared absorption band that is smaller than the threshold as an immobilized lipase having a lipase activity, or as an immobilized lipase capable of reactivating a part or the entire lipase activity.

The present invention can be further considered to be a method for evaluating the peroxidase activity of an immobilized peroxidase formed by immobilizing a peroxidase on a silica carrier, wherein the evaluation method has an evaluation process comprising: a step of approximating an infrared absorption band derived from a peroxidase appearing around 1500 to 1600 cm⁻¹ (absorption band II) or an infrared absorption band derived from a peroxidase appearing around 1600 to 1700 cm⁻¹ (absorption band I) in the infrared absorption spectrum of the immobilized peroxidase by one or more normal distributions, a step of calculating an index value indicating a degree of broadening of the infrared absorption band based on the normal distributions, and a step of comparing the index value with a predetermined threshold, and evaluating an immobilized peroxidase having a degree of broadening of the infrared absorption band that is smaller than the threshold as an immobilized peroxidase having a peroxidase activity.

The present invention can be further considered to be a method for evaluating the activity of an antibody, wherein the evaluation method has an evaluation process comprising: a step of approximating an infrared absorption band derived from an antibody appearing around 1500 to 1600 cm⁻¹ (absorption band II) or an infrared absorption band derived from an antibody appearing around 1600 to 1700 cm⁻¹ (absorption band I) in the infrared absorption spectrum of the antibody by one or more normal distributions, a step of calculating an index value indicating a degree of broadening of the infrared absorption band based on the normal distributions, and a step of comparing the index value with a predetermined threshold, and evaluating an antibody having a degree of broadening of the infrared absorption band that is smaller than the threshold as an antibody having an activity.

The present invention also relates to a method for producing a compound that is produced via a catalytic reaction of using a lipase, in which the immobilized lipase or the reactivated immobilized lipase of the present invention is used. Since the immobilized lipase or the reactivated immobilized lipase has a sufficient catalytic activity or is capable of reactivating a sufficient catalytic activity, the production efficiency of the compound is improved.

The compound may be produced via a transesterification reaction or an ester hydrolysis reaction. Moreover, the compound may be a polycarbonate diol (meth)acrylate compound.

### Effect of the Invention

According to the present invention, a method for producing a protein, by which a protein having a sufficient activity can be obtained, and a protein obtained by this production method, can be provided. In addition, according to the present invention, a method for evaluating the activity of a protein, which can evaluate the activity of a protein without requiring the actual measurement of the activity, can be provided.

According to the present invention, a method for producing an immobilized lipase, by which the reversible denaturation of a catalytic activity can be distinguished from the irreversible denaturation thereof, and by which an immobilized lipase having a sufficient catalytic activity can be obtained, and an immobilized lipase obtained by this production method, can be provided.

Moreover, according to the present invention, a method for producing a reactivated immobilized lipase, by which the reversible denaturation of a catalytic activity can be distinguished from the irreversible denaturation thereof, and by which a reactivated immobilized lipase having a sufficient catalytic activity can be obtained, and a reactivated immobilized lipase obtained by this production method, can be provided.

Furthermore, according to the present invention, a method for evaluating the activity of a lipase, which can distinguish an immobilized lipase having a lipase activity or an immobilized lipase possibly reactivating a part or the entire lipase activity from among other immobilized lipases, without requiring the actual measurement of the activity, can be provided.

Since the immobilized lipase or the reactivated immobilized lipase of the present invention has a sufficient catalytic activity or is capable of reactivating a sufficient catalytic activity, it is preferably used for production of a compound that is produced via a catalytic reaction of using a lipase.

According to the present invention, a method for producing an immobilized peroxidase, by which the reversible denaturation of a catalytic activity can be distinguished from the irreversible denaturation thereof, and by which an immobilized peroxidase having a sufficient catalytic activity can be obtained, and an immobilized peroxidase obtained by the production method, can be provided.

According to the present invention, a method for evaluating the activity of a peroxidase, which can distinguish an immobilized peroxidase having a peroxidase activity or an immobilized peroxidase possibly reactivating a part or the entire peroxidase activity from among other immobilized peroxidases, without requiring the actual measurement of the activity, can be provided.

According to the present invention, a method for producing an antibody, by which an antibody having a sufficient titer can be obtained, and an antibody obtained by this production method, can be provided.

According to the present invention, a method for evaluating the activity of an antibody, which can evaluate the titer of an antibody without requiring the actual measurement of the activity, can be provided.

### BRIEF DESCRIPTION OF DRAWINGS

FIG. 1 is an explanatory diagram for explaining a method of calculating an absorption band area ratio.

FIG. 2 is a view showing an example of approximating an infrared absorption band derived from a lipase (absorption band I) by a single normal distribution.

FIG. 3 is a view showing an example of subjecting an infrared absorption band derived from a lipase (absorption band I) to waveform separation to obtain two normal distributions.

FIG. 4 is a view showing an example of subjecting an infrared absorption band derived from a lipase (absorption band I) to waveform separation to obtain three normal distributions.

FIG. 5 is a view showing an example of subjecting an infrared absorption band derived from a lipase (absorption band I) to waveform separation to obtain eight normal distributions.

FIG. 6 is a view showing an example of subjecting an infrared absorption band derived from a lipase (absorption band I) to waveform separation to obtain eight normal distributions.

FIG. 7 is a view showing an example of subjecting an infrared absorption band derived from a lipase (absorption band I) to waveform separation to obtain eight normal distributions.

FIG. 8 is a view showing an example of subjecting an infrared absorption band derived from a lipase (absorption band I) to waveform separation to obtain eight normal distributions.

FIG. 9 is a graph showing a correlation of index value 1 (half-value width) with the specific activity of a lipase in a transesterification reaction.

FIG. 10 is a graph showing a correlation of index value 1 (half-value width) with index value 7 (half-value width).

FIG. 11 is a graph showing a correlation of index value 1 (half-value width) with the specific activity of a lipase in a transesterification reaction.

FIG. 12 is a graph showing a correlation of index value 2 (absorption band area ratio) with the specific activity of a lipase in a transesterification reaction.

FIG. 13 is a graph showing a correlation of index value 3 (absorption band area ratio) with the specific activity of a lipase in a transesterification reaction.

FIG. 14 is a graph showing a correlation of index value 4 (absorption band area ratio) with the specific activity of a lipase in a transesterification reaction.

FIG. 15 is a graph showing a correlation of index value 5 (absorption band area ratio) with the specific activity of a lipase in a transesterification reaction.

FIG. 16 is a graph showing a correlation of index value 6 (absorption band area ratio) with the specific activity of a lipase in a transesterification reaction.

FIG. 17 is a view showing an example of approximating an infrared absorption band derived from a peroxidase (absorption band II) by a single normal distribution.

FIG. 18 is a view showing an example of subjecting an infrared absorption band derived from a peroxidase (absorption band II) to waveform separation to obtain three normal distributions.

FIG. 19 is a graph showing a correlation of index value 7 (half-value width) with the specific activity of a peroxidase in an oxidation reaction.

FIG. 20 is a graph showing a correlation of index value 8 (absorption band area ratio) with the specific activity of a peroxidase in an oxidation reaction.

FIG. 21 is an example of approximating infrared absorption bands derived from an antibody (absorption band I and absorption band II) by a single normal distribution.

FIG. 22 is a view showing an example of subjecting an infrared absorption band derived from an antibody to waveform separation to obtain eight normal distributions (absorption band I) and an example of subjecting an infrared absorption band derived from an antibody to waveform separation to obtain three normal distributions (absorption band II)

FIG. 23 is a graph showing the results obtained by measuring the titer of an antibody, on which various treatments have been carried out.

FIG. 24 is a graph showing a correlation of index value 1 (half-value width) with the titer of an antibody.

FIG. 25 is a graph showing a correlation of index value 6 (absorption band area ratio) and index value 8 (absorption band area ratio) with the titer of an antibody.

### EMBODIMENTS FOR CARRYING OUT THE INVENTION

Hereinafter, the embodiments for carrying out the present invention will be described in detail. However, the present invention is not limited to the following embodiments.

[Method for Producing a Protein]

The method for producing a protein according to the present embodiment comprises at least an inspection process. This inspection process comprises a step of approximating an infrared absorption band derived from a protein appearing around 1500 to 1600 cm⁻¹ (absorption band II) or an infrared absorption band derived from a protein appearing around 1600 to 1700 cm⁻¹ (absorption band I) in the infrared absorption spectrum of the protein by one or more normal distributions, a step of calculating an index value indicating a degree of broadening of the infrared absorption band based on the normal distributions, and a step of comparing the index value with a predetermined threshold to select, as a good-quality product, a protein having a degree of broadening of the infrared absorption band that is smaller than the threshold.

The protein produced by the method for producing a protein according to the present embodiment is not particularly limited, and it may be any given protein. On the other hand, according to the method for producing a protein according to the present embodiment, since a protein having an activity or a protein capable of reactivating an activity can be selected in the inspection process, the protein to be produced is preferably a functional protein. The functional protein herein means a protein having an activity when it has not undergone denaturation or the like. In addition, in the present description, the “activity” includes a catalytic activity, a binding activity to a receptor or the like, and the titer of an antibody.

Examples of such a protein having an activity can include: enzymes that are proteins having a catalytic activity, such as lipase, peroxidase or protease; proteins having a binding activity to a receptor, which can also be used as pharmaceutical products, such as interferon, erythropoietin or insulin; and antibodies.

The protein to be produced may be a protein immobilized on a carrier. It is to be noted that the “carrier” described in the present description includes: resin carriers (e.g., an ion exchange resin) and inorganic carriers (e.g., a silica carrier), which are capable of retaining proteins by adsorption or the like; thickeners to be mixed with proteins; and solvents and solutes in protein solutions.

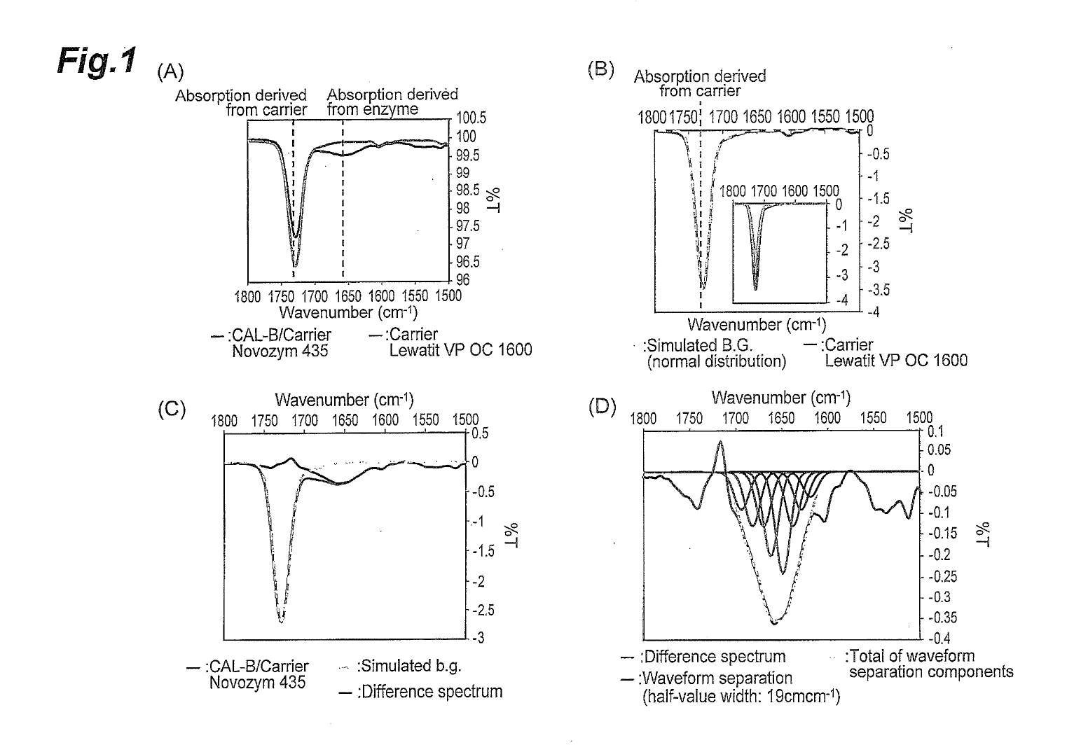

The index value according to the present embodiment indicates the degree of broadening of absorption band I or absorption band II. FIG. 1 is an explanatory diagram for explaining a method of calculating an index value (which is herein an absorption band area ratio). Hereafter, a method of calculating an index value will be described, with reference to FIG. 1. It is to be noted that the following calculation method is merely an example, and that it is possible to make various modifications.

First, the infrared absorption spectrum of a protein is measured. Herein, when the protein is a protein immobilized on a carrier, the infrared absorption spectrum of the carrier itself is also measured. It may also be possible that the infrared absorption spectrum of the carrier itself has previously been measured. As methods of measuring an infrared absorption spectrum, a transmission method, a diffuse reflection method, and an attenuated total reflection method (ATR method) are preferable, and from the viewpoint of measuring an absorption spectrum derived from a trace amount of protein with high sensitivity, the ATR method is more preferable.

FIG. 1(A) shows an infrared absorption spectrum obtained in a case where Novozym (registered trademark) 435, in which CalB is immobilized on an immobilized lipase Lewatit (registered trademark) VP OC 1600, is used as a protein immobilized on a carrier, and the infrared absorption spectrum of the resin carrier (Lewatit (registered trademark) VP OC 1600) itself. The difference spectrum between the two spectra can be an infrared absorption spectrum derived from the protein (lipase). However, in order to calculate a difference between individually measured infrared absorption spectra, there is a case where absorption intensity needs to be determined.

Next, the infrared absorption spectrum derived from the carrier is simulated. That is to say, the infrared absorption spectrum derived from the carrier is approximated by a normal distribution, and it can be used as a background spectrum for the infrared absorption spectrum of a protein immobilized on an individually measured carrier. Specifically, by changing intensity, the individually measured infrared absorption spectrum of the protein immobilized on the carrier is fitted into the infrared absorption spectrum derived from the carrier. FIG. 1(B) shows an example of simulation of the infrared absorption spectrum derived from the carrier.

Subsequently, a difference spectrum is calculated from the infrared absorption spectrum of the protein immobilized on the carrier and the infrared absorption spectrum derived from the carrier (simulation), and an infrared absorption spectrum derived from the protein in the protein immobilized on the carrier is extracted. FIG. 1(C) shows an example of calculation of a difference spectrum.

Calculation of a difference spectrum can be carried out, for example, by applying a Gaussian function and by using a spreadsheet program (e.g., Microsoft Excel; manufactured by Microsoft).

Next, by using the difference spectrum, absorption band I or II is approximated by one or more normal distributions.

Approximation of the difference spectrum by the single normal distribution can be carried out, for example, by a non-linear least-squares method. The non-linear least-squares method can be carried out by utilizing the Solver function of a spreadsheet program (e.g., Microsoft Excel; manufactured by Microsoft), and by setting, for example, a peak position, a half-value width and intensity, as variable numbers.

The index value in a case where it is approximated by a single normal distribution can be, for example, a half-value width (unit: cm⁻¹) of the normal distribution. In this case, as the index value (half-value width) decreases, the degree of broadening of the infrared absorption band decreases.

Approximation of the difference spectrum by a plurality of normal distributions can be carried out, for example by subjecting it to waveform separation to obtain a plurality of normal distributions. Waveform separation can be carried out, for example, by applying a Gaussian function and by using a spreadsheet program (e.g., Microsoft Excel; manufactured by Microsoft). Specifically, when the spectrum is subjected to waveform separation to obtain an n number of normal distributions A₁ to Aₙ, the waveform separation can be carried out by utilizing the Solver function, specifying the band positions of the wavenumbers of A₁ to Aₙ and the default value of the half-value width, and making a calculation according to the non-linear least-squares method, so that a sum of squared residuals between a sum of the areas of A₁ to Aₙ and the area of the absorption band of the difference spectrum becomes a minimum. The band positions of the wavenumbers of A₁ to Aₙ may be assigned at equal intervals, or at different intervals. In addition, the band position of an identical wavenumber may also be assigned to at least two of A₁ to Aₙ.

The number of normal distributions to be subjected to waveform separation (namely, the value of n) is not particularly limited, and it may be set, as appropriate, depending on purpose. The number n is preferably 2 to 20, and more preferably 2, 3, 5 or 8, since the precision for selecting a protein as a good-quality product can be further improved.

In FIG. 1 for example, a difference spectrum is used, and an infrared absorption band derived from a protein (lipase) appearing around 1600 to 1700 cm⁻¹ is subjected to waveform separation to obtain eight normal distributions A₁, A₂, A₃, A₄, A₅, A₆, A₇ and A₈ (which are collectively also referred to as A₁ to A₈). A₁ to A₈ are eight normal distributions, in which the peak positions are adjacent to one another at intervals of approximately 10 cm⁻¹, and the half-value width is approximately 19 cm⁻¹. Moreover, waveform separation is carried out, such that a sum of the areas of A₁ to A₈ (a total of waveform separation components) becomes as equal as possible to the area of the absorption band of the difference spectrum. FIG. 1(D) shows an example of waveform separation.

In A₁ to A₈ in FIG. 1, the band positions of wavenumbers are set at 8 points, namely, 1692 cm⁻¹ (A₁), 1682 cm⁻¹ (A₂), 1670 cm⁻¹ (A₃), 1658 cm⁻¹ (A₄), 1648 cm⁻¹ (A₅), 1638 cm⁻¹ (A₆), 1629 cm⁻¹ (A₇) and 1619 cm⁻¹ (A₈), as default values in the Gaussian function used in fitting, and each half-value width is set at 19 cm⁻¹.

The index value in a case where the infrared absorption band is subjected to waveform separation to obtain two normal distributions can be, for example, a value obtained by subjecting the infrared absorption band to waveform separation to obtain two normal distributions each having a peak around the peak top position of the infrared absorption band and having a different half-value width, and then dividing the area of a normal distribution having a smaller half-value width among the two normal distributions by the area of a normal distribution having a larger half-value width. The band positions of the waveforms of the two normal distributions may be identical to each other. In this case, as the index value increases, the degree of broadening of the infrared absorption band decreases.

The index value in a case where the infrared absorption band is approximated by a plurality of (three or more) normal distributions can be a value obtained by dividing a sum of the areas of one or more normal distributions around the peak top position of the infrared absorption band by a sum of the areas of one or more normal distributions around the end of the infrared absorption band (absorption band area ratio). In this case, as the index value increases, the degree of broadening of the infrared absorption band decreases.

When the infrared absorption band is subjected to waveform separation to obtain an n number of normal distributions A₁ to Aₙ (wherein n is an integer of 3 or greater), the index value can be calculated, for example, as follows.
(i) When the number n is an even number, the index value is defined as a value obtained by dividing a sum of the area(s) of at least one or both of A_{n/2} and A_{n/2+1} by a sum of the area of at least one selected from the group consisting of A₁ to A_{n/2-1} and A_{n/2+2} to Aₙ.
(ii) When the number n is an odd number, the index value is defined as a value by dividing the area of A_{(n+1)/2} by a sum of the area of at least one selected from the group consisting of A₁ to A_{(n+1)/2-1} and A_{(n-1)/2+2} to Aₙ.

For example, when the number n is 3, the index value can be A₂/(A₁+A₃). For example, when the number n is 5, the index value may be A₃/(A₁+A₂+A₃+A₄), or A₃/(A₁+A₄), or A₃/(A₁+A₃+A₄), or A₃/(A₁+A₂+A₄). In addition, when the number n is an odd number of 5 or greater, the index value calculated in the above (ii) may be, for example, a value obtained by dividing a sum of the area of at least one selected from the group consisting of A_{(n+1)/2-1} to A_{(n-1)/2+2} by a sum of the area of at least one selected from the group consisting of A₁ to A_{(n+1)/2-2} and A_{(n-1)/2+3} to Aₙ.

For example, when the number n is 8, the index value may be (A₄+A₅)/(A₂+A₃+A₈) or (A₄+A₅)/(A₁+A₂+A₃+A₆+A₇+A₈). Moreover, when the number n is an even number of 6 or greater, the index value calculated in the above (i) may be, for example, a value obtained by dividing a sum of the area of at least one selected from the group consisting of A_{n/2-1} to A_{n/2+2} by a sum of the area of at least one selected from the group consisting of A₁ to A_{n/2-2} and A_{n/2+3} to Aₙ.

The threshold used to determine whether or not the protein is a good-quality product may be set, as appropriate, depending on the type of a protein to be produced, the activity level required for a protein to be produced, etc. Since the index value as calculated above has a linearly approximatable correlation with the activity of a protein or the potential activity of a protein, the threshold can be set depending on the activity level required for a protein to be produced. In addition, in order to determine a threshold, it is preferable to previously analyze the correlation between the index value and the activity regarding a protein to be produced.

An example of the threshold will be described. In the case of the immobilized lipase shown in FIG. 1, when the index value is (A₄+A₅)/(A₂+A₃+A₈), an immobilized lipase, in which the index value (absorption band area ratio) is 1.2 (threshold) or more, can be evaluated as an immobilized lipase having a lipase activity, or as an immobilized lipase possibly reactivating a part or the entire lipase activity. The threshold may be set at 1.3, or 1.4, or 1.5. The index value is generally approximately 5.0 at a maximum.

The above-explained method of calculating an index value does not depend on the type of a protein. That is, even if the protein is, for example, a lipase, a peroxidase, an antibody and the like, the index value can be calculated in the same manner as that describe above.

The method for producing a protein according to the present embodiment may comprise a process of synthesizing a protein, in addition to the aforementioned inspection process. For the synthesis of a protein, a known means can be used. For instance, yeasts, filamentous fungi, animal cells, animals, or the like, into which DNA encoding the protein has been incorporated for expression, are cultured or bred, so that they are allowed to express the protein, and the expressed protein is then recovered (purified) from a disrupted cell product, a medium, animal milk, or the like, thereby synthesizing the protein.

Since the protein obtained by the method for producing a protein according to the present embodiment has undergone the aforementioned inspection process, it has a sufficient activity or is capable of reactivating a sufficient activity, and it can be preferably used for industrial use, medicinal use, and the like.

[Method for Evaluating the Activity of a Protein]

The method for evaluating the activity of a protein according to the present embodiment has an evaluation process comprising: a step of approximating absorption band I or absorption band II in the infrared absorption spectrum of the protein by one or more normal distributions, a step of calculating an index value indicating a degree of broadening of the infrared absorption band based on the normal distributions, and a step of comparing the index value with a predetermined threshold to evaluate, as a protein having an activity, a protein having a degree of broadening of the infrared absorption band that is smaller than the threshold.

Examples of the index value and the threshold applied in the method for evaluating the activity of a protein according to the present embodiment include the same as those in the above described method for producing an immobilized lipase.

[Method for Producing an Immobilized Lipase]

In the present description, the immobilized lipase means a lipase that is immobilized on a resin carrier according to adsorption or the like.

The lipase may be an enzyme that catalyzes a hydrolysis reaction of an ester bond. Moreover, the lipase is preferably an enzyme that also catalyzes an ester synthesis reaction. Specific examples of such an enzyme include a cutinase derived from Cryptococcus sp., a lipase derived from Burkholderia cepacia (e.g., Amano PS (manufactured by Amano Enzyme Inc.)), a lipase derived from Candida antarctica (e.g., Novozym 435 (manufactured by Novozymes)), a lipase derived from Rhizomucor Miehei, a lipase derived from Thermomyces lanuginosus (e.g., Lipase TL (manufactured by Meito Sangyo Co., Ltd.)), and a lipase derived from Mucor Miehei. Among these, a lipase derived from Burkholderia cepacia and a lipase derived from Candida antarctica are preferable.

The above described lipase may be obtained by obtaining a gene encoding the lipase from the above described microorganisms, transforming a suitable host such as yeast or filamentous fungi with the obtained gene, and then obtaining the lipase from a culture of the obtained genetically recombinant form. The recombination DNA technology used for the recombinant expression of the lipase is well known in the present technical field.

The lipase may also be a mutant of a lipase derived from the above described microorganisms. For instance, it may be a lipase comprising a deletion, substitution or addition of one or several amino acids in the amino acid sequence of the lipase derived from the above described microorganisms and having at least a hydrolysis activity on an ester bond. Moreover, it may also be a lipase showing a sequence identity of 90% or more, preferably 95% or more, and more preferably 97% or more at the amino acid sequence level with the lipase derived from the above described microorganisms and having at least a hydrolysis activity on an ester bond.

From the viewpoint that the adsorption amount of the lipase can be increased when the resin carrier has a large surface area, the resin carrier is preferably a porous resin carrier consisting of a porous body. Examples of the resin carrier include organic polymers such as an ion exchange resin, a hydrophobic absorption resin, a chelate resin, and a synthetic adsorption resin. Among these, from the viewpoint of having a particularly high powder of adsorbing enzyme, a hydrophobic adsorption resin is preferable.

As such a resin carrier, a resin carrier that is commonly used for immobilization of enzyme can be used. Specifically, polystyrene, polyacrylic acid ester, polypropylene, polyethylene, and polyamide can be used, for example. These substances may be copolymers or may be crosslinked. Among others, a polystyrene-copolymer and a poly-(meth)acrylic acid ester (for example, polymethyl methacrylate crosslinked with divinyl benzene) are preferable. These resin carriers are macroporous, and typically have a modal small pore diameter of approximately 5 to 20 nm and a total surface area of 50 to 1000 m²/g (according to a nitrogen adsorption method).

A commercially available hydrophobic adsorption resin may be used. Specific examples include Lewatit (registered trademark) VP OC 1600 (manufactured by LANXESS, Germany), Amberlite (registered trademark) XAD-7HP (manufactured by Organo Corporation, Japan), and DIAION HP20 (manufactured by Mitsubishi Chemical Corporation, Japan).

Immobilization of a lipase on a resin carrier can be carried out, for example, by carrier binding methods involving a covalent bond, an ionic bond, physical adsorption, etc., and inclusion methods comprising immobilizing a lipase on a lattice of a resin having a network structure. Among others, a carrier binding method involving physical adsorption is preferable because this method is simple.

Immobilization by physical adsorption can be carried out, for example, by allowing an aqueous solution of a lipase to come into contact with a resin carrier. Moreover, the immobilization method may also comprise separating an immobilized lipase that has been formed by adsorption of the lipase on the resin carrier from the water phase, then washing the separated immobilized lipase, and then drying it to result in an appropriate water content percentage.

The temperature and the pH applied upon immobilization by physical adsorption may be set, as appropriate, depending on the type of a lipase to be immobilized. Many types of lipases can be immobilized, for example, at a room temperature and at a pH close to a neutral range. As a time required for the contact of the resin carrier with the lipase, in general, a time required for essentially complete adsorption is selected. This is typically from 1 or 2 hours to 24 hours.

When an immobilized lipase is used for a continuous transesterification in a fixed-bed column, the immobilized lipase is preferably composed of spherical particles having a uniform particle diameter. The particle diameter is preferably 100 to 5000 μm, and more preferably 300 to 1000 μm.

The method for producing an immobilized lipase according to the present embodiment has an inspection process comprising: a step of approximating absorption band I or absorption band II in the infrared absorption spectrum of the immobilized lipase by one or more normal distributions, a step of calculating an index value indicating a degree of broadening of the infrared absorption band based on the normal distributions, and a step of comparing the index value with a predetermined threshold, and selecting, as a good-quality product, an immobilized lipase having a degree of broadening of the infrared absorption band that is smaller than the threshold.

In the method for producing an immobilized lipase according to the present embodiment, an immobilized lipase, in which when the index value is index value 1, the index value becomes 70 cm⁻¹ (threshold) or less, is preferably selected as a good-quality product. The threshold is more preferably 65 cm⁻¹. The index value is, in general, approximately 55 cm⁻¹ at a minimum.

In the method for producing an immobilized lipase according to the present embodiment, an immobilized lipase, in which when the index value is index value 2, the index value becomes 0.27 (threshold) or more, is preferably selected as a good-quality product. The threshold is more preferably 0.49. The index value is, in general, approximately 1.00 at a maximum.

In the method for producing an immobilized lipase according to the present embodiment, an immobilized lipase, in which when the index value is index value 3, the index value becomes 0.9 (threshold) or more, is preferably selected as a good-quality product. The threshold is more preferably 1.5. The index value is, in general, approximately 1.9 at a maximum.

In the method for producing an immobilized lipase according to the present embodiment, an immobilized lipase, in which when the index value is index value 4, the index value becomes 0.35 (threshold) or more, is preferably selected as a good-quality product. The threshold is more preferably 0.40. The index value is, in general, approximately 0.49 at a maximum.

In the method for producing an immobilized lipase according to the present embodiment, an immobilized lipase, in which when the index value is index value 5, the index value becomes 0.6 (threshold) or more, is preferably selected as a good-quality product. The threshold is more preferably 0.65. The index value is, in general, approximately 0.70 at a maximum.

In the method for producing an immobilized lipase according to the present embodiment, an immobilized lipase, in which when the index value is index value 6, the index value becomes 1.2 (threshold) or more, is preferably selected as a good-quality product. The threshold is more preferably 1.3. The index value is, in general, approximately 1.4 at a maximum.

In the method for producing an immobilized lipase according to the present embodiment, an immobilized lipase, in which when the index value is index value 7, the index value becomes 44 cm⁻¹ (threshold) or less, is preferably selected as a good-quality product. The threshold is more preferably 40 cm⁻¹. The index value is, in general, approximately 35 cm⁻¹ at a minimum.

In the method for producing an immobilized lipase according to the present embodiment, an immobilized lipase, in which when the index value is index value 8, the index value becomes 1.2 (threshold) or more, is preferably selected as a good-quality product. The threshold is more preferably 1.5. The index value is, in general, approximately 1.7 at a maximum.

The above described method for producing an immobilized lipase may comprise a process of obtaining an immobilized lipase formed by immobilizing a lipase on a resin carrier according to a conventional method. The immobilized lipases obtained by this process may include those having a low catalytic activity due to irreversible denaturation or reversible denaturation; however, it becomes possible to distinguish such irreversible denaturation from reversible denaturation by performing the above described inspection process.

In the case of reversible denaturation, the catalytic activity is apparently low, but the catalytic activity can be reactivated, for example, by adjusting a water content percentage.

[Method for Producing a Reactivated Immobilized Lipase]

In the method for producing a reactivated immobilized lipase according to the present embodiment, a reactivated immobilized lipase, in which a part or the entire lipase activity is reactivated, is produced from an immobilized lipase having a reduced lipase activity. This production method has a selection process comprising: a step of approximating absorption band I or absorption band II in the infrared absorption spectrum of the immobilized lipase having a reduced lipase activity by one or more normal distributions, a step of calculating an index value indicating a degree of broadening of the infrared absorption band based on the normal distributions, and a step of comparing the index value with a predetermined threshold, and selecting, as an immobilized lipase in which a lipase activity is possibly reactivated, the immobilized lipase having a reduced lipase activity that has a degree of broadening of the infrared absorption band that is smaller than the threshold.

Examples of the index value and the threshold applied in the method for producing a reactivated immobilized lipase according to the present embodiment include the same as those in the above described method for producing an immobilized lipase.

Since the index value is used as an index in the selection process, a reversibly denaturable immobilized lipase can be selected from among immobilized lipases having a reduced lipase activity. Accordingly, the immobilized lipase selected in the selection process is capable of reactivating a catalytic activity, for example, by adjusting a water content percentage.

The above described reactivated method for producing an immobilized lipase may comprise a reactivation process for treating the immobilized lipase having a reduced lipase activity selected in the selection process with water or a hydrous organic solvent. By performing the reactivation process, the immobilized lipase can be adjusted to have an appropriate water content percentage, and thus, the lipase activity is reactivated. The reason why the lipase activity can be reactivated is that an immobilized lipase having a reduced lipase activity that has a degree of broadening of the infrared absorption band that is smaller than the threshold has been selected in the selection process.

Examples of the water that can be used herein include ultrapure water, distilled water, ion exchange water, tap water, and industrial water. From the viewpoint of avoiding the mixing of inorganic salts, among these waters, it is preferable to use ion exchange water and distilled water. As a hydrous organic solvent, a solvent prepared by adding the aforementioned water to an organic solvent such as acetone, ethanol, acetonitrile or 1-butanol can be used. The water content percentage of such a hydrous organic solvent is different depending on the type of the organic solvent, and when acetone is used as an organic solvent for example, the water content percentage can be set at 1.0% or more.

The activation process can be carried out, for example, by allowing the immobilized lipase selected in the selection process to come into contact with water or a hydrous organic solvent. This contact can be carried out, for example, in a temperature range of −10° C. to 50° C. for a time range of 0.5 to 48 hours, by appropriately setting the water content percentage of the immobilized lipase as an index. The water content percentage of the immobilized lipase used as an index is different depending on the type of a lipase, the amount of the lipase immobilized, the type of a resin carrier and the like, and when the immobilized lipase is, for example, 3.4 wt % Amano PS/Lewatit, the water content percentage is 5% or more, and when the immobilized lipase is Novozym (registered trademark) 435, the water content percentage is 0.05% or more.

[Method for Evaluating the Lipase Activity of an Immobilized Lipase]

The method for evaluating the lipase activity of an immobilized lipase according to the present embodiment has an evaluation process comprising: a step of approximating absorption band I or absorption band II in the infrared absorption spectrum of the immobilized lipase by one or more normal distributions, a step of calculating an index value indicating a degree of broadening of the infrared absorption band based on the normal distributions, and a step of comparing the index value with a predetermined threshold, and evaluating an immobilized lipase having a degree of broadening of the infrared absorption band that is smaller than the threshold as an immobilized lipase having a lipase activity, or as an immobilized lipase capable of reactivating a part or the entire lipase activity.

Examples of the index value and the threshold applied in the method for evaluating the lipase activity of an immobilized lipase according to the present embodiment include the same as those in the above described method for producing an immobilized lipase.

[Method for Producing a Compound]

The method for producing a compound according to the present embodiment is characterized in that it uses the immobilized lipase obtained by the above described method for producing an immobilized lipase, or the reactivated immobilized lipase obtained by the above described method for producing a reactivated immobilized lipase. The compound is produced via a catalytic reaction using a lipase.

The compound is preferably produced by performing a transesterification reaction or an ester hydrolysis reaction, and examples of the compound include fuel for fuel diesel prepared by a transesterification reaction of oils and fats with alcohol, and a polycarbonate diol (meth)acrylate compound. Among these compounds, a polycarbonate diol (meth)acrylate compound is more preferable.

[Method for Producing an Immobilized Peroxidase]

In the present description, the immobilized peroxidase means a peroxidase immobilized on a silica carrier by adsorption or the like.

The peroxidase may be an enzyme that catalyzes a reaction of oxidatively cleaving peroxide (—O—O—) and decomposing it into two hydroxyl groups. Specific examples of the peroxidase include a peroxidase derived from horseradish (horseradish peroxidase) (e.g., HRP (manufactured by Wako Pure Chemical Industries, Ltd.)), a cytochrome c peroxidase, and a glutathione peroxidase. Among these, peroxidase derived from horseradish is preferable.

The above described peroxidase may be obtained by obtaining a gene encoding the peroxidase, transforming a suitable host such as yeast or filamentous fungi with the obtained gene, and then obtaining the peroxidase from a culture of the obtained genetically recombinant form. The recombination DNA technology used for the recombinant expression of the peroxidase is well known in the present technical field.

The peroxidase may also be a mutant of the peroxidase. For instance, it may be a peroxidase comprising a deletion, substitution or addition of one or several amino acids in the amino acid sequence of the peroxidase and having at least an activity of oxidatively cleaving peroxide and decomposing it into two hydroxyl groups. Moreover, it may also be a peroxidase showing an amino acid sequence identity of 90% or more, preferably 95% or more, and more preferably 97% or more with the above described peroxidase and having at least an activity of oxidatively cleaving peroxide and decomposing it into two hydroxyl groups.

From the viewpoint that the adsorption amount of the peroxidase can be increased because a silica carrier has a large surface area, the silica carrier is preferably a porous silica carrier consisting of a porous body. Examples of the silica carrier include mesoporous silica such as MCFs, FSM-16, MCM-41, MCM-48 and SBA-15. Among these, from the viewpoint that the adsorption amount of enzyme is particularly large, MCFs is preferable.

Immobilization of a peroxidase on a silica carrier can be carried out, for example, by a covalent bond, an ionic bond, a carrier binding method involving physical adsorption. Among others, a carrier binding method involving physical adsorption is preferable because this method is simple.

Immobilization by physical adsorption can be carried out, for example, by allowing an aqueous solution of a peroxidase to come into contact with a silica carrier. Moreover, the immobilization method may also comprise separating an immobilized peroxidase that has been formed by adsorption of the peroxidase on the silica carrier from the water phase, then washing the separated immobilized peroxidase, and then drying it to result in an appropriate water content percentage.

The temperature and the pH applied upon immobilization by physical adsorption may be set, as appropriate, depending on the type of a peroxidase to be immobilized. Many types of peroxidases can be immobilized, for example, at a room temperature and at a pH close to a neutral range. As a time required for the contact of the silica carrier with the peroxidase, in general, a time required for essentially complete adsorption is selected. This is typically from 1 or 2 hours to 24 hours.

The method for producing an immobilized peroxidase according to the present embodiment has an inspection process comprising: a step of approximating absorption band I or absorption band II in the infrared absorption spectrum of the immobilized peroxidase by one or more normal distributions, a step of calculating an index value indicating a degree of broadening of the infrared absorption band based on the normal distributions, and a step of comparing the index value with a predetermined threshold, and selecting, as a good-quality product, an immobilized peroxidase having a degree of broadening of the infrared absorption band that is smaller than the threshold.

In the method for producing an immobilized peroxidase according to the present embodiment, an immobilized peroxidase, in which when the index value is index value 7, the index value becomes 75 cm⁻¹ (threshold) or less, is preferably selected as a good-quality product. The threshold is more preferably 70 cm⁻¹. The index value is, in general, approximately 65 cm⁻¹ at a minimum.

In the method for producing an immobilized peroxidase according to the present embodiment, an immobilized peroxidase, in which when the index value is index value 8, the index value becomes 0.45 (threshold) or more, is preferably selected as a good-quality product. The threshold is more preferably 0.50. The index value is, in general, approximately 0.70 at a maximum.

The method for producing an immobilized peroxidase according to the present embodiment may comprise a process of obtaining an immobilized peroxidase formed by immobilizing a peroxidase on a silica carrier according to a conventional method. The immobilized peroxidases obtained by this process may include those having a low catalytic activity due to irreversible denaturation or reversible denaturation; however, it becomes possible to distinguish such irreversible denaturation from reversible denaturation by performing the above described inspection process.

In the case of reversible denaturation, the catalytic activity is apparently low, but the catalytic activity can be reactivated, for example, by adjusting a water content percentage.

[Method for Evaluating the Activity of a Peroxidase]

The method for evaluating the peroxidase activity of an immobilized peroxidase according to the present embodiment has an evaluation process comprising: a step of approximating absorption band I or absorption band II in the infrared absorption spectrum of the immobilized peroxidase by one or more normal distributions, a step of calculating an index value indicating a degree of broadening of the infrared absorption band based on the normal distributions, and a step of comparing the index value with a predetermined threshold to evaluate, as an immobilized peroxidase having an peroxidase activity, an immobilized peroxidase having a degree of broadening of the infrared absorption band that is smaller than the threshold.

Examples of the index value and the threshold applied in the method for evaluating the peroxidase activity of an immobilized peroxidase according to the present embodiment include the same as those in the above described method for producing an immobilized peroxidase.

[Method for Producing an Antibody]

In the present description, the antibody may be any one of IgG, IgM, IgA, IgD, and IgE. In addition, the present antibody may also be a fragment of an antibody having an antigen-binding ability (e.g., scFV, F(ab), F(ab′)₂, etc.). The antibody may also be a human antibody, a humanized antibody, a chimeric antibody, a mouse antibody, a rabbit antibody, or a chicken antibody.

The method for producing an antibody according to the present embodiment has an inspection process comprising: a step of approximating absorption band I or absorption band II in the infrared absorption spectrum of the antibody by one or more normal distributions, a step of calculating an index value indicating a degree of broadening of the infrared absorption band based on the normal distributions, and a step of comparing the index value with a predetermined threshold, and selecting, as a good-quality product, an antibody having a degree of broadening of the infrared absorption band that is smaller than the threshold.

In the method for producing an antibody according to the present embodiment, an antibody, in which when the index value is index value 1, the index value becomes 65 cm⁻¹ (threshold) or less, is preferably selected as a good-quality product. The threshold is more preferably 60 cm⁻¹. The index value is, in general, approximately 57 cm⁻¹ at a minimum.

In the method for producing an antibody according to the present embodiment, an antibody, in which when the index value is index value 6, the index value becomes 0.98 (threshold) or more, is preferably selected as a good-quality product. The threshold is more preferably 1.00. The index value is, in general, approximately 1.09 at a maximum.

In the method for producing an antibody according to the present embodiment, an antibody, in which when the index value is index value 8, the index value becomes 0.85 (threshold) or more, is preferably selected as a good-quality product. The threshold is more preferably 0.90. The index value is, in general, approximately 0.97 at a maximum.

The method for producing an antibody according to the present embodiment may, comprise a process of obtaining an antibody according to a conventional method. This process can be carried out, for example, by culturing or breeding yeasts, filamentous fungi, animal cells, animals, or the like, into which DNA encoding the antibody has been incorporated for expression, so that they are allowed to express the antibody, and then by recovering (purifying) the expressed antibody from a disrupted cell product, a medium, animal milk, or the like, thereby producing an antibody having a sufficient titer.

[Method for Evaluating the Activity of an Antibody]

The method for evaluating the activity of an antibody according to the present embodiment has an evaluation process comprising: a step of approximating absorption band I or absorption band II in the infrared absorption spectrum of the antibody by one or more normal distributions, a step of calculating an index value indicating a degree of broadening of the infrared absorption band based on the normal distributions, and a step of comparing the index value with a predetermined threshold, and evaluating an antibody having a degree of broadening of the infrared absorption band that is smaller than the threshold as an antibody having an activity. Herein, the activity of an antibody is, for example, the titer of an antibody.

Examples of the index value and the threshold applied in the method for evaluating the activity of an antibody according to the present embodiment include the same as those in the above described method for producing an antibody.

### EXAMPLES

Hereinafter, the present invention will be specifically described in the following test examples, however, the scope of the present invention is not limited to these examples.

[1. Evaluation of the Catalytic Activity of an Immobilized Lipase]

### <Method of Quantifying Hexyl Acrylate>

Hexyl acrylate was quantified by gas chromatography (internal standard method). Measurement conditions for gas chromatography were as follows.
Gas chromatography equipment (GC2014, manufactured by Shimadzu Corporation)
Analysis column: DB-5 (30 m×0.53 mm, ID: 1.0 μm, manufactured by Shimadzu Corporation)
Column temperature: 60° C.→retained for 2 minutes→10° C./min→200° C.→retained for 2 minutes
Injection port temperature: 200° C.
Detection port temperature (FID): 250° C.
Carrier gas: helium
Linear velocity: 29.4 cm/sec
Split ratio: 1:50
Amount injected: 1.0 μL
Retention time

Hexyl acrylate: 9.31 minutes

Tetraethylene glycol dimethyl ether (internal standard substance): 15.6 minutes

<Method of Calculating a Specific Activity in a Transesterification Reaction>

Using the amount of the generated hexyl acrylate that had been quantified by the above described method, the specific activity of an immobilized lipase formed by immobilizing a lipase on each porous resin carrier in a transesterification reaction was calculated according to the following expression.

Specific activity in transesterification reaction (mmol·h⁻¹·g⁻¹)=(Amount of hexyl acrylate generated (mmol))/(Lipase weight (g)×reaction time (h)) [Expression 1]

<Method of Measuring a Water Content Percentage>

Using a moisture meter (MOC63u, dry weight method, manufactured by Shimadzu Corporation), approximately 1.0 g of sample was subjected to two repeated measurements under measurement conditions of a standard dry automatic stop mode (120° C., water content change percentage: 0.05%), and the mean value was then calculated.

<Method of Measuring an FT-IR Spectrum>

The FT-IR spectrum of each immobilized lipase was measured by using a Fourier transform infrared spectrophotometer (FTS7000e microscope UMA600; manufactured by Agilent Technologies).

### Test Example 1

### Calculation of Index Values

### <Absorption Band I>

Index values 1 to 6 were calculated from absorption band I derived from a lipase appearing around 1600 to 1700 cm⁻¹ in an FT-IR spectrum according to the following procedures.

(Index Value 1: Half-Value Width in Single Normal Distribution Approximation)

With regard to each immobilized lipase, the obtained FT-IR spectrum was approximated by a single normal distribution by using a spreadsheet program (Microsoft Excel; manufactured by Microsoft). When the FT-IR spectrum was approximated by the single normal distribution, first, baseline correction was carried out, so that the infrared absorption intensity at 1800 and 1575 cm⁻¹ could be 0. Subsequently, an absorption band derived from a porous resin carrier appearing around 1720 cm⁻¹ was separated by using a Gaussian function. Thereafter, an absorption band derived from a lipase appearing around 1660 cm⁻¹ was approximated by a single normal distribution according to a non-linear least-squares method, and a half-value width was then calculated as an index value. It is to be noted that, in the non-linear least-squares method, a peak position, a half-value width and intensity were set at variable numbers.

(Index Value 2: Absorption Band Area Ratio Obtained by Subjecting an FT-IR Spectrum to Waveform Separation to Obtain Two Normal Distributions)

With regard to each immobilized lipase, the obtained FT-IR spectrum was subjected to waveform separation by using a spreadsheet program (Microsoft Excel; manufactured by Microsoft), and the absorption band area ratio was obtained. When the FT-IR spectrum was subjected to waveform separation, first, baseline correction was carried out, so that the infrared absorption intensity at 1800 and 1575 cm⁻¹ could be 0. Subsequently, an absorption band derived from a porous resin carrier appearing around 1720 cm⁻¹ was separated by using a Gaussian function, and thereafter, an absorption band derived from a lipase appearing around 1660 cm⁻¹ was subjected to waveform separation. As a default value in the Gaussian function that was to be used for fitting, the band position of the wavenumber was set at one point that was 1656 cm⁻¹ (A₁ and A₂), and the half-value widths were set at 47 cm⁻¹ (A₁) and 82 cm⁻¹ (A₂). Each absorption band was separated by a non-linear least-squares method, and by using the obtained area of each absorption band, the absorption band area ratio was calculated as index value 2 according to the following formula:

Absorption band area ratio (index value 2)=A₁/A₂,

wherein A₁ and A₂ each represent the area of each absorption band.

(Index Value 3: Absorption Band Area Ratio Obtained by Subjecting an FT-IR Spectrum to Waveform Separation to Obtain Three Normal Distributions)

With regard to each immobilized lipase, the obtained FT-IR spectrum was subjected to waveform separation by using a spreadsheet program (Microsoft Excel; manufactured by Microsoft), and the absorption band area ratio was obtained. When the FT-IR spectrum was subjected to waveform separation, first, baseline correction was carried out, so that the infrared absorption intensity at 1800 and 1575 cm⁻¹ could be 0. Subsequently, an absorption band derived from a porous resin carrier appearing around 1720 cm⁻¹ was separated by using a Gaussian function, and thereafter, an absorption band derived from a lipase appearing around 1660 cm⁻¹ was subjected to waveform separation. As default values in the Gaussian function that were to be used for fitting, the band positions of the wavenumbers were set at three points that were 1680 cm⁻¹ (A₁), 1656 cm⁻¹ (A₂) and 1631 cm⁻¹ (A₃), and their half-value width was set at 50 cm⁻¹. Each absorption band was separated by a non-linear least-squares method, and by using the obtained area of each absorption band, the absorption band area ratio was calculated as index value 3 according to the following formula:

Absorption band area ratio (index value 3)=A₂/(A₁+A₃),

wherein A₁, A₂ and A₃ each represent the area of each absorption band.

(Index Value 4: Absorption Band Area Ratio Obtained by Subjecting an FT-IR Spectrum to Waveform Separation to Obtain Five Normal Distributions)

With regard to each immobilized lipase, the obtained FT-IR spectrum was subjected to waveform separation by using a spreadsheet program (Microsoft Excel; manufactured by Microsoft), and the absorption band area ratio was obtained. When the FT-IR spectrum was subjected to waveform separation, first, baseline correction was carried out, so that the infrared absorption intensity at 1800 and 1575 cm⁻¹ could be 0. Subsequently, an absorption band derived from a porous resin carrier appearing around 1720 cm⁻¹ was separated by using a Gaussian function, and thereafter, an absorption band derived from a lipase appearing around 1660 cm⁻¹ was subjected to waveform separation. As default values in the Gaussian function that were to be used for fitting, the band positions of the wavenumbers were set at five points that were 1685 cm⁻¹ (A₁), 1670 cm⁻¹ (A₂), 1656 cm⁻¹ (A₃), 1641 cm⁻¹ (A₄) and 1626 cm⁻¹ (A₅), and their half-value width was set at 30 cm⁻¹. Each absorption band was separated by a non-linear least-squares method, and by using the obtained area of each absorption band, the absorption band area ratio was calculated as index value 4 according to the following formula:

Absorption band area ratio (index value 4)=A₃/(A₁+A₂+A₄+A₅),

wherein A₁, A₂, A₃, A₄ and A₅ each represent the area of each absorption band.

(Index Value 5 and Index Value 6: Absorption Band Area Ratio Obtained by Subjecting an FT-IR Spectrum to Waveform Separation to Obtain Eight Normal Distributions)

With regard to each immobilized lipase, the obtained FT-IR spectrum was subjected to waveform separation by using a spreadsheet program (Microsoft Excel; manufactured by Microsoft), and the absorption band area ratio was obtained. When the FT-IR spectrum was subjected to waveform separation, first, baseline correction was carried out, so that the infrared absorption intensity at 1800 and 1575 cm⁻¹ could be 0. Subsequently, an absorption band derived from a porous resin carrier appearing around 1720 cm⁻¹ was separated by using a Gaussian function, and thereafter, an absorption band derived from a lipase appearing around 1660 cm⁻¹ was subjected to waveform separation. As default values in the Gaussian function that were to be used for fitting, the band positions of the wavenumbers were set at eight points that were 1692 cm⁻¹ (A₁), 1682 cm⁻¹ (A₂), 1670 cm⁻¹ (A₃), 1658 cm⁻¹ (A₄), 1648 cm⁻¹ (A₅), 1638 cm⁻¹ (A₆), 1629 cm⁻¹ (A₇) and 1619 cm⁻¹ (A₈), and their half-value width was set at 19 cm⁻¹. Each absorption band was separated by a non-linear least-squares method, and by using the obtained area of each absorption band, the absorption band area ratios were calculated as index value 5 and index value 6 according to the following formulae:

Absorption band area ratio (index value 5)=(A₄+A₅)/(A₁+A₂+A₃+A₆+A₇+A₈),

wherein A₁, A₂, A₃, A₄, A₅, A₆, A₇ and A₈ each represent the area of each absorption band; and

Absorption band area ratio (index value 6)=(A₄+A₅)/(A₂+A₃+A₈),

wherein A₂, A₃, A₄, A₅ and A₈ each represent the area of each absorption band.

<Absorption Band II>

Index values 7 and 8 were calculated from absorption band II derived from a lipase appearing around 1500 to 1600 cm⁻¹ in an FT-IR spectrum according to the following procedures.

(Index Value 7: Half-Value Width in Single Normal Distribution Approximation)

With regard to each immobilized lipase, the obtained FT-IR spectrum was approximated by a single normal distribution by using a spreadsheet program (Microsoft Excel; manufactured by Microsoft). When the FT-IR spectrum was approximated by the single normal distribution, first, baseline correction was carried out, so that the infrared absorption intensity at 1800 and 1575 cm⁻¹ could be 0. Subsequently, an absorption band derived from a porous resin carrier appearing around 1400 to 1500 cm⁻¹ was separated by using a Gaussian function. Thereafter, an absorption band derived from a lipase appearing around 1500 to 1570 cm⁻¹ was approximated by a single normal distribution according to a non-linear least-squares method, and a half-value width was then calculated as an index value. It is to be noted that, in the non-linear least-squares method, a peak position, a half-value width and intensity were set at variable numbers.

(Index Value 8: Absorption Band Area Ratio Obtained by Subjecting an FT-IR Spectrum to Waveform Separation to Obtain Three Normal Distributions)

With regard to each immobilized lipase, the obtained FT-IR spectrum was subjected to waveform separation by using a spreadsheet program (Microsoft Excel; manufactured by Microsoft), and the absorption band area ratio was obtained. When the FT-IR spectrum was subjected to waveform separation, first, baseline correction was carried out, so that the infrared absorption intensity at 1800 and 1575 cm⁻¹ could be 0. Subsequently, an absorption band derived from a porous resin carrier appearing around 1400 to 1500 cm⁻¹ was separated by using a Gaussian function, and thereafter, an absorption band derived from a lipase appearing around 1500 to 1570 cm⁻¹ was subjected to waveform separation. As default values in the Gaussian function that were to be used for fitting, the band positions of the wavenumbers were set at three points that were 1570 cm⁻¹ (B₁), 1545 cm⁻¹ (B₂), 1518 cm⁻¹ (B₃), and their half-value width was set at 31 cm⁻¹. Each absorption band was separated by a non-linear least-squares method, and by using the obtained area of each absorption band, the absorption band area ratio was calculated as index value 8 according to the following formula:

Absorption band area ratio (index value 8)=B₂/(B₁+B₃),

wherein B₁, B₂ and B₃ each represent the area of each absorption band.

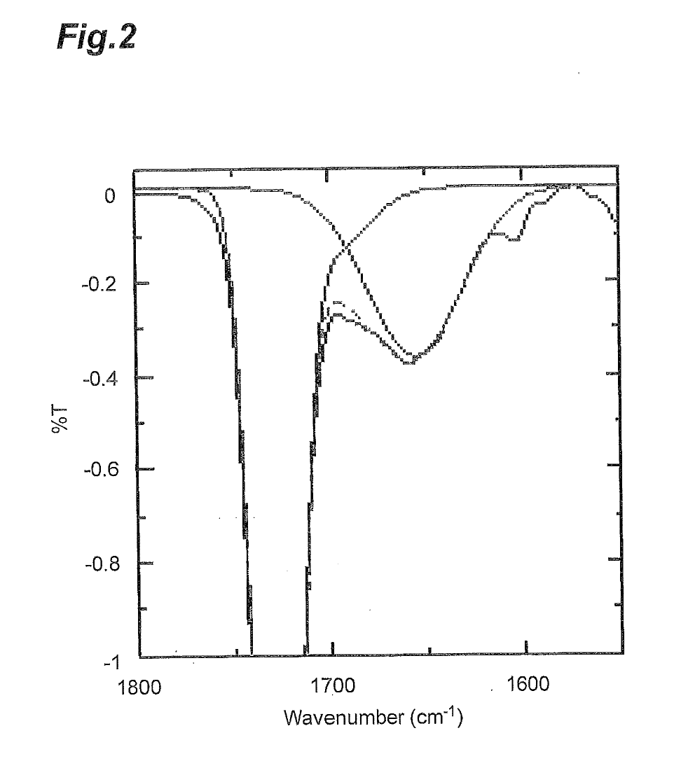

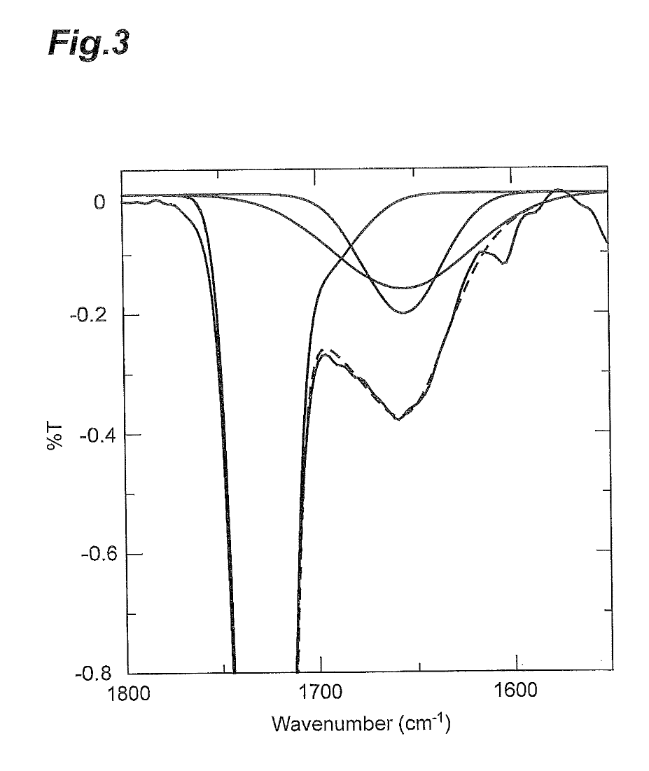

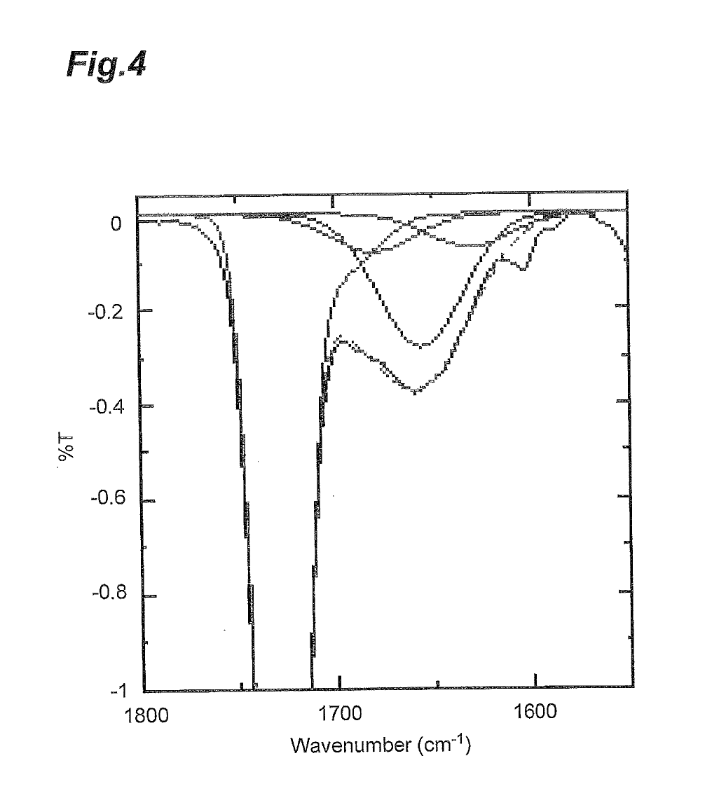

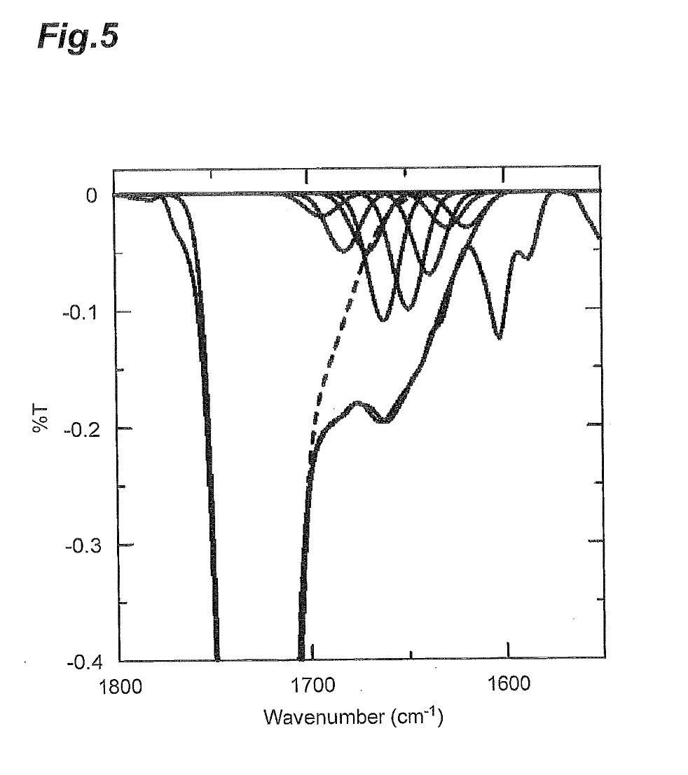

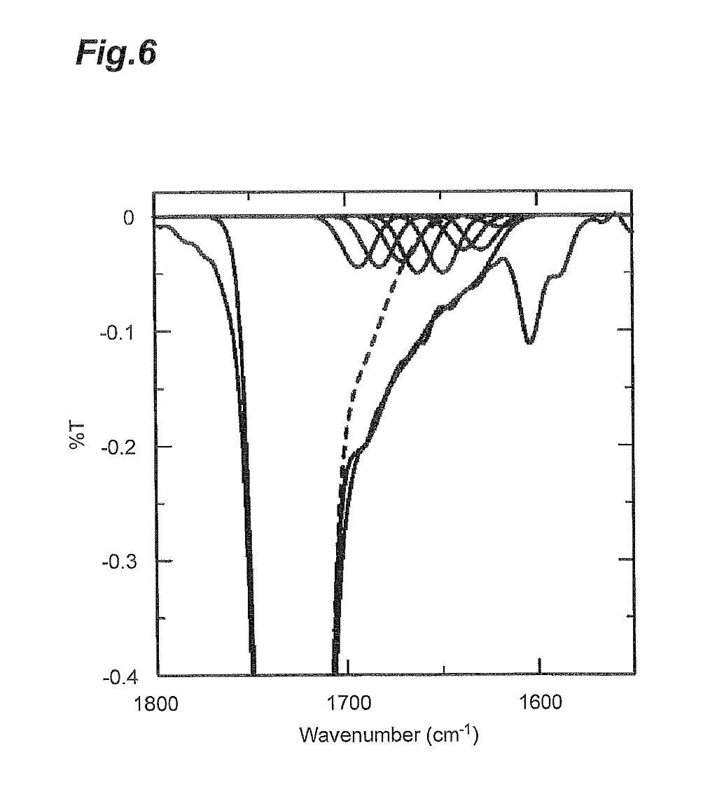

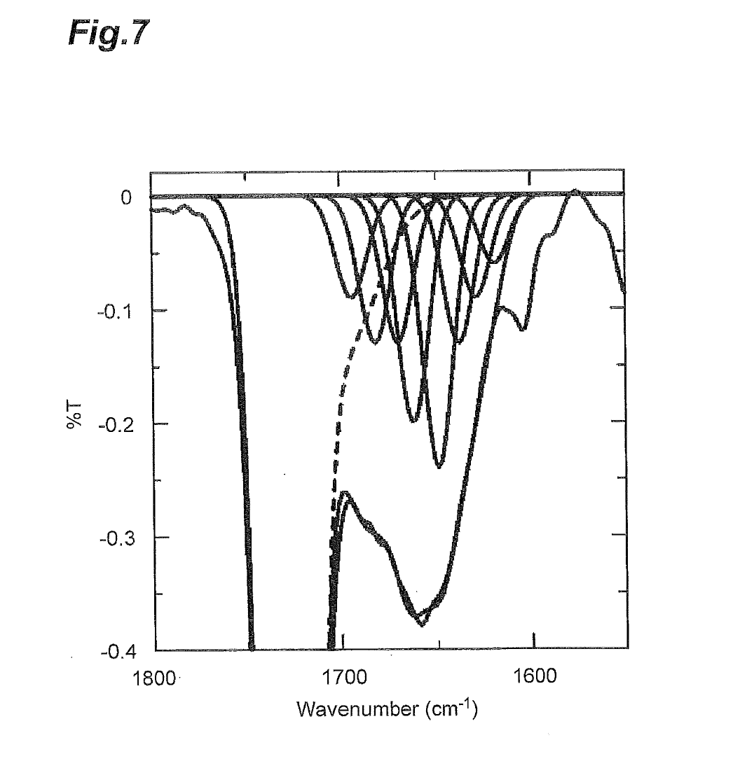

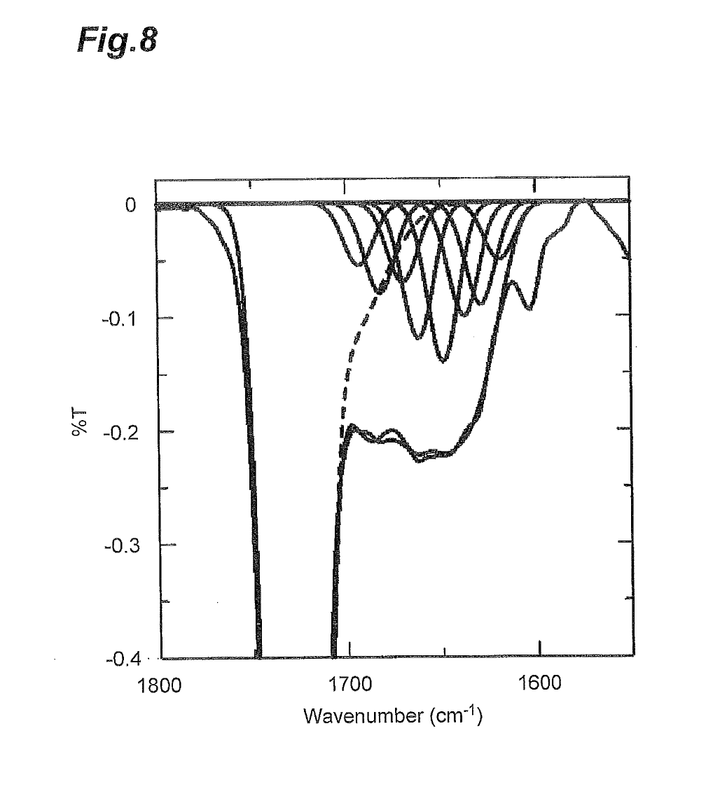

FIGS. 2 to 8 show an example of approximating an infrared absorption band derived from a lipase by a single normal distribution and examples of subjecting such an infrared absorption band derived from a lipase to waveform separation. FIG. 2 shows the FT-IR spectrum of an immobilized lipase (Novozym 435) used as a catalyst in Test Example 17, and an example of approximating absorption band I derived from the lipase by a single normal distribution. FIG. 3 shows the FT-IR spectrum of an immobilized lipase (Novozym 435) used as a catalyst in Test Example 17, and an example of subjecting absorption band I derived from the lipase to waveform separation to obtain two normal distributions. FIG. 4 shows the FT-IR spectrum of an immobilized lipase (Novozym 435) used as a catalyst in Test Example 17, and an example of subjecting absorption band I derived from the lipase to waveform separation to obtain three normal distributions. FIG. 5 shows the FT-IR spectrum of an immobilized lipase (3.4 wt % Amano PS/Lewatit) used as a catalyst in Test Example 3, and a graph showing individual bands obtained by subjecting absorption band I derived from the lipase to waveform separation to obtain eight normal distributions. FIG. 6 shows the FT-IR spectrum of an immobilized lipase (3.4 wt % Amano PS/Lewatit) used as a catalyst in Test Example 11, and a graph showing individual bands obtained by subjecting absorption band I derived from the lipase to waveform separation to obtain eight normal distributions. FIG. 7 shows the FT-IR spectrum of an immobilized lipase (Novozym 435) used as a catalyst in Test Example 14, and a graph showing individual bands obtained by subjecting absorption band I derived from the lipase to waveform separation to obtain eight normal distributions. FIG. 8 shows the FT-IR spectrum of an immobilized lipase (Novozym 435) used as a catalyst in Test Example 15, and a graph showing individual bands obtained by subjecting absorption band I derived from the lipase to waveform separation to obtain eight normal distributions.

### Test Example 2

### Preparation of Immobilized Lipase PS (3.4 wt % Amano PS/Lewatit)

0.6 L of 100 mM phosphate buffer (pH 7.0) and pure water were added to 150 g of lipase PS “Amano” SDH (manufactured by Amano Enzyme Inc.) so that it was dissolved therein, and thereafter, the resulting solution was adjusted to a constant volume (3 L), thereby preparing an immobilized solution. To the solution, 600 g of a porous resin carrier (LEWATIT VPOC 1600, manufactured by LANXESS) was added, and the obtained mixture was then stirred at 6° C. for 12 hours. Thereafter, the carrier was recovered by decantation, was then washed with pure water, and was then dried under a reduced pressure, to obtain 352 g of an immobilized lipase (the amount of the lipase immobilized: 3.4 wt %). This immobilized lipase was referred to as an “immobilized lipase PS” or “3.4 wt % Amano PS/Lewatit.”

It is to be noted that, with regard to the amount of the lipase immobilized, the concentration of a protein in a supernatant after completion of the immobilization, relative to BSA, was measured according to a BCA method, and a decrease in the protein concentrations obtained before and after the immobilization was defined as the amount of the protein immobilized on the carrier, so that the total amount of the protein immobilized was calculated.

### Test Example 3

### Synthesis of Hexyl Acrylate

To a glass test tube having an internal volume of 20 mL that was equipped with a stirrer and a temperature-control device, 0.2 g (2.0 mmol) of 1-hexanol, 1.6 g (18.6 mmol) of methyl acrylate, 0.04 g (0.2 mmol) of tetraethylene glycol dimethyl ether, and immobilized lipase PS having a water content percentage of 7.1% used as a catalyst were added, and the obtained mixture was then reacted at 40° C., while being stirred. One hour later, the reaction solution was removed, and the specific activity in the transesterification reaction was then calculated according to the aforementioned method. The specific activity of the lipase in the transesterification reaction was 259.3 mmol·h⁻¹·g⁻¹. In addition, index value 1 (absorption band I, half-value width) obtained by the aforementioned method was 54.1 cm⁻¹, and index value 6 (absorption band I, absorption band area ratio—eight normal distributions) was 1.75.

### Test Example 4

### Synthesis of Hexyl Acrylate

The same reaction as that in Test Example 3 was carried out, with the exception that immobilized lipase PS having a water content percentage of 1.0%, was used as a catalyst. The specific activity of the lipase in the transesterification reaction was 16.5 mmol·h⁻¹·g⁻¹. In addition, index value 1 (absorption band I, half-value width) obtained by the aforementioned method was 54.1 cm⁻¹, and index value 6 (absorption band I, absorption band area ratio—eight normal distributions) was 1.73.

### Test Example 5

### Synthesis of Hexyl Acrylate

The same reaction as that in Test Example 3 was carried out, with the exception that immobilized lipase PS prepared by immersing it in methanol at 25° C. for 2 hours, and then washing it with purified water to adjust the water content percentage thereof to 71.0%, was used as a catalyst. The specific activity of the lipase in the transesterification reaction was 89.8 mmol·h⁻¹·g⁻¹. In addition, index value 1 (absorption band I, half-value width) obtained by the aforementioned method was 6.1.1 cm⁻¹, and index value 6 (absorption band I, absorption band area ratio—eight normal distributions) was 1.46.

### Test Example 6

### Synthesis of Hexyl Acrylate

The same reaction as that in Test Example 3 was carried out, with the exception that immobilized lipase PS prepared by immersing it in toluene at 25° C. for 2 hours, and then washing it with purified water to adjust the water content percentage thereof to 50.0%, was used as a catalyst. The specific activity of the lipase in the transesterification reaction was 216.1 mmol·h⁻¹·g⁻¹. In addition, index value 1 (absorption band I, half-value width) obtained by the aforementioned method was 54.1 cm⁻¹, and index value 6 (absorption band I, absorption band area ratio—eight normal distributions) was 1.68.

### Test Example 7

### Synthesis of Hexyl Acrylate

The same reaction as that in Test Example 3 was carried out, with the exception that immobilized lipase PS prepared by immersing it in acetone at 25° C. for 2 hours, and then washing it with purified water to adjust the water content percentage thereof to 52.4%, was used as a catalyst. The specific activity of the lipase in the transesterification reaction was 169.7 mmol·h⁻¹·g⁻¹. In addition, index value 1 (absorption band I, half-value width) obtained by the aforementioned method was 61.1 cm⁻¹, and index value 6 (absorption band I, absorption band area ratio—eight normal distributions) was 1.44.

### Test Example 8

### Synthesis of Hexyl Acrylate

The same reaction as that in Test Example 3 was carried out, with the exception that immobilized lipase PS prepared by immersing it in acetonitrile at 25° C. for 2 hours, and then washing it with purified water to adjust the water content percentage thereof to 58.6%, was used as a catalyst. The specific activity of the lipase in the transesterification reaction was 127.4 mmol·h⁻¹·g⁻¹. In addition, index value 1 (absorption band I, half-value width) obtained by the aforementioned method was 63.5 cm⁻¹, and index value 6 (absorption band I, absorption band area ratio—eight normal distributions) was 133.

### Test Example 9

### Synthesis of Hexyl Acrylate

The same reaction as that in Test Example 3 was carried out, with the exception that immobilized lipase PS prepared by immersing it in 1-butanol at 25° C. for 2 hours, and then washing it with purified water to adjust the water content percentage thereof to 65.5%, was used as a catalyst. The specific activity of the lipase in the transesterification reaction was 79.7 mmol·h⁻¹·g⁻¹. In addition, index value 1 (absorption band I, half-value width) obtained by the aforementioned method was 65.8 cm⁻¹, and index value 6 (absorption band I, absorption band area ratio—eight normal distributions) was 1.38.

### Test Example 10

### Synthesis of Hexyl Acrylate

The same reaction as that in Test Example 3 was carried out, with the exception that immobilized lipase PS prepared by immersing it in ethanol at 25° C. for 2 hours, and then washing it with purified water to adjust the water content percentage thereof to 60.2%, was used as a catalyst. The specific activity of the lipase in the transesterification reaction was 87.0 mmol·h⁻¹·g⁻¹. In addition, index value 1 (absorption band I, half-value width) obtained by the aforementioned method was 65.8 cm⁻¹, and index value 6 (absorption band I, absorption band area ratio—eight normal distributions) was 1.24.

### Test Example 11

### Synthesis of Hexyl Acrylate

The same reaction as that in Test Example 3 was carried out, with the exception that immobilized lipase PS prepared by retaining it in the atmosphere at 230° C. for 1 hour, and then washing it with purified water to adjust the water content percentage thereof to 40.0%, was used as a catalyst. Hexyl acrylate as a product was not observed. In addition, index value 1 (absorption band I, half-value width) obtained by the aforementioned method was 70.5 cm⁻¹, and index value 6 (absorption band I, absorption band area ratio—eight normal distributions) was 0.95.

### Test Example 12

### Synthesis of Hexyl Acrylate

The same reaction as that in Test Example 3 was carried out, with the exception that immobilized lipase PS prepared by using it in a fixed bed flow type transesterification reaction for 3 months and then washing it with toluene was used as a catalyst. The specific activity of the lipase in the transesterification reaction was 13.6 mmol·h⁻¹·g⁻¹. In addition, index value 1 (absorption band I, half-value width) obtained by the aforementioned method was 61.1 cm⁻¹, and index value 6 (absorption band I, absorption band area ratio—eight normal distributions) was 1.28.

### Test Example 13

### Synthesis of Hexyl Acrylate

The same reaction as that in Test Example 3 was carried out, with the exception that immobilized lipase PS prepared by using it in the same fixed bed flow type transesterification reaction as that in Example 12 for 3 months, then washing it with toluene, and then immersing it in acetone having a water content percentage of 5% at 6° C. overnight to adjust the water content percentage thereof to 46.1%, was used as a catalyst. The specific activity of the lipase in the transesterification reaction was 88.2 mmol·h⁻¹·g⁻¹. In addition, index value 1 (absorption band I, half-value width) obtained by the aforementioned method was 61.1 cm⁻¹, and index value 6 (absorption band I, absorption band area ratio—eight normal distributions) was 1.33.

### Test Example 14

### Synthesis of Hexyl Acrylate

The same reaction as that in Test Example 3 was carried out, with the exception that immobilized lipase PS prepared by immersing it in an acetic acid aqueous solution (pH 2) at 25° C. for 2 hours, and then washing it with purified water to adjust the water content percentage thereof to 25.0%, was used as a catalyst. The specific activity of the lipase in the transesterification reaction was 209.6 mmol·h⁻¹·g⁻¹. In addition, index value 1 (absorption band I, half-value width) obtained by the aforementioned method was 56.4 cm⁻¹, and index value 6 (absorption band I, absorption band area ratio—eight normal distributions) was 1.55.

### Test Example 15

### Synthesis of Hexyl Acrylate

The same reaction as that in Test Example 3 was carried out, with the exception that immobilized lipase PS prepared by immersing it in a hydrochloric acid aqueous solution (pH 4) at 25° C. for 16 hours, and then washing it with purified water to adjust the water content percentage thereof to 12.0%, was used as a catalyst. The specific activity of the lipase in the transesterification reaction was 220.5 mmol·h⁻¹·g⁻¹. In addition, index value 1 (absorption band I, half-value width) obtained by the aforementioned method was 61.1 cm⁻¹, and index value 6 (absorption band I, absorption band area ratio—eight normal distributions) was 1.51.

### Test Example 16

### Synthesis of Hexyl Acrylate

The same reaction as that in Test Example 3 was carried out, with the exception that immobilized lipase PS prepared by immersing it in a hydrochloric acid aqueous solution (pH 4) at 70° C. for 16 hours, and then washing it with purified water to adjust the water content percentage thereof to 33.0%, was used as a catalyst. The specific activity of the lipase in the transesterification reaction was 56.1 mmol·h⁻¹·g⁻¹. In addition, index value 1 (absorption band I, half-value width) obtained by the aforementioned method was 65.8 cm⁻¹, and index value 6 (absorption band I, absorption band area ratio—eight normal distributions) was 1.21.

### Test Example 17

### Synthesis of Hexyl Acrylate

The same reaction as that in Test Example 3 was carried out, with the exception that Novozym (registered trademark) 435 (manufactured by Novozymes) having a water content percentage of 1.0% was used as a catalyst. The specific activity of the lipase in the transesterification reaction was 346.4 mmol·h⁻¹·g⁻¹. In addition, index value 1 (absorption band I, half-value width) obtained by the aforementioned method was 57.6 cm⁻¹, index value 2 (absorption band I, absorption band area ratio—two normal distributions) was 0.72, index value 3 (absorption band I, absorption band area ratio—three normal distributions) was 1.87, index value 4 (absorption band I, absorption band area ratio—five normal distributions) was 0.49, index value 5 (absorption band I, absorption band area ratio—eight normal distributions) was 0.70, index value 6 (absorption band I, absorption band area ratio—eight normal distributions) was 1.42, index value 7 (absorption band II, half-value width) was 37. 6 cm⁻¹, and index value 8 (absorption band II, absorption band area ratio—three normal distributions) was 1.75. It is to be noted that Novozym (registered trademark) 435 is an immobilized lipase in which lipase CalB is immobilized on a porous resin carrier.

### Test Example 18

### Synthesis of Hexyl Acrylate

The same reaction as that in Test Example 3 was carried out, with the exception that Novozym (registered trademark) 435 prepared by immersing it in methanol at 25° C. for 3 hours, and then adjusting the water content percentage thereof to 1.0% by vacuum drying, was used as a catalyst. The specific activity of the lipase in the transesterification reaction was 98.6 mmol·h⁻¹·g⁻¹. In addition, index value 1 (absorption band I, half-value width) obtained by the aforementioned method was 70.5 cm⁻¹, index value 2 (absorption band I, absorption band area ratio—two normal distributions) was 0.26, index value 3 (absorption band I, absorption band area ratio—three normal distributions) was 0.88, index value 4 (absorption band I, absorption band area ratio—five normal distributions) was 0.31, index value 5 (absorption band I, absorption band area ratio—eight normal distributions) was 0.58, index value 6 (absorption band I, absorption band area ratio—eight normal distributions) was 1.06, index value 7 (absorption band II, half-value width) was 44.7 cm⁻¹, and index value 8 (absorption band II, absorption band area ratio—three normal distributions) was 1.13.

### Test Example 19

### Synthesis of Hexyl Acrylate

The same reaction as that in Test Example 3 was carried out, with the exception that Novozym (registered trademark) 435 prepared by immersing it in toluene at 25° C. for 3 hours, and then adjusting the water content percentage thereof to 1.0% by vacuum drying, was used as a catalyst. The specific activity of the lipase in the transesterification reaction was 392.6 mmol·h⁻¹·g⁻¹. In addition, index value 1 (absorption band I, half-value width) obtained by the aforementioned method was 56.4 cm⁻¹, index value 2 (absorption band I, absorption band area ratio—two normal distributions) was 0.92, index value 3 (absorption band I, absorption band area ratio—three normal distributions) was 2.10, index value 4 (absorption band I, absorption band area ratio—five normal distributions) was 0.50, index value 5 (absorption band I, absorption band area ratio—eight normal distributions) was 0.69, and index value 6 (absorption band I, absorption band area ratio—eight normal distributions) was 1.48.

### Test Example 20

### Synthesis of Hexyl Acrylate

The same reaction as that in Test Example 3 was carried out, with the exception that Novozym (registered trademark) 435 prepared by immersing it in acetone at 25° C. for 3 hours, and then adjusting the water content percentage thereof to 1:0% by vacuum drying, was used as a catalyst. The specific activity of the lipase in the transesterification reaction was 384.4 mmol·h⁻¹·g⁻¹. In addition, index value 1 (absorption band I, half-value width) obtained by the aforementioned method was 56.4 cm⁻¹, index value 2 (absorption band I, absorption band area ratio—two normal distributions) was 1.05, index value 3 (absorption band I, absorption band area ratio—three normal distributions) was 2.50, index value 4 (absorption band I, absorption band area ratio—five normal distributions) was 0.53, index value 5 (absorption band I, absorption band area ratio—eight normal distributions) was 0.73, and index value 6 (absorption band I, absorption band area ratio—eight normal distributions) was 1.58.

### Test Example 21

### Synthesis of Hexyl Acrylate

The same reaction as that in Test Example 3 was carried out, with the exception that Novozym (registered trademark) 435 prepared by immersing it in acetonitrile at 25° C. for 3 hours, and then adjusting the water content percentage thereof to 1.0% by vacuum drying, was used as a catalyst. The specific activity of the lipase in the transesterification reaction was 375.9 mmol·h⁻¹·g⁻¹. In addition, index value 1 (absorption band I, half-value width) obtained by the aforementioned method was 56.4 cm⁻¹, index value 2 (absorption band I, absorption band area ratio—two nonnal distributions) was 0.88, index value 3 (absorption band I, absorption band area ratio—three normal distributions) was 2.20, index value 4 (absorption band I, absorption band area ratio—five normal distributions) was 0.48, index value 5 (absorption band I, absorption band area ratio—eight normal distributions) was 0.74, and index value 6 (absorption band I, absorption band area ratio—eight normal distributions) was 1.55.

### Test Example 22

### Synthesis of Hexyl Acrylate

The same reaction as that in Test Example 3 was carried out, with the exception that Novozym (registered trademark) 435 prepared by retaining it in the atmosphere at 230° C. for 1 hour, and then adjusting the water content percentage thereof to 0.1%, was used as a catalyst. Hexyl acrylate as a product was not observed. In addition, index value 1 (absorption band I, half-value width) obtained by the aforementioned method was 82.3 cm⁻¹, index value 2 (absorption band I, absorption band area ratio—two normal distributions) was 0, index value 3 (absorption band I, absorption band area ratio—three normal distributions) was 0.47, index value 4 (absorption band I, absorption band area ratio—five normal distributions) was 0.25, index value 5 (absorption band I, absorption band area ratio—eight normal distributions) was 0.45, index value 6 (absorption band I, absorption band area ratio—eight normal distributions) was 0.85, index value 7 (absorption band II, half-value width) was 47.0 cm⁻¹, and index value 8 (absorption band II, absorption band area ratio—three normal distributions) was 0.58.

### Test Example 23

### Synthesis of Hexyl Acrylate

The same reaction as that in Test Example 3 was carried out, with the exception that Novozym (registered trademark) 435 prepared by immersing it in an acetic acid aqueous solution (pH 2) at 25° C. for 2 hours, and then adjusting the water content percentage thereof to 9.0% by vacuum drying, was used as a catalyst. The specific activity of the lipase in the transesterification reaction was 224.2 mmol·h⁻¹·g⁻¹. In addition, index value 1 (absorption band I, half-value width) obtained by the aforementioned method was 58.8 cm⁻¹, index value 2 (absorption band I, absorption band area ratio—two normal distributions) was 0.59, index value 3 (absorption band I, absorption band area ratio—three normal distributions) was 1.63, index value 4 (absorption band I, absorption band area ratio—five normal distributions) was 0.45, index value 5 (absorption band I, absorption band area ratio—eight normal distributions) was 0.66, and index value 6 (absorption band I, absorption band area ratio—eight normal distributions) was 1.39.

### Test Example 24

### Synthesis of Hexyl Acrylate

The same reaction as that in Test Example 3 was carried out, with the exception that Novozym (registered trademark) 435 prepared by immersing it in a hydrochloric acid aqueous solution (pH 4) at 25° C. for 16 hours, and then adjusting the water content percentage thereof to 3.0% by vacuum drying, was used as a catalyst. The specific activity of the lipase in the transesterification reaction was 351.6 mmol·h⁻¹·g⁻¹. In addition, index value 1 (absorption band I, half-value width) obtained by the aforementioned method was 58.8 cm⁻¹, index value 2 (absorption band I, absorption band area ratio—two normal distributions) was 0.55, index value 3 (absorption band I, absorption band area ratio—three normal distributions) was 1.97, index value 4 (absorption band I, absorption band area ratio—five normal distributions) was 0.50, index value 5 (absorption band I, absorption band area ratio—eight normal distributions) was 0.70, and index value 6 (absorption band I, absorption band area ratio—eight normal distributions) was 1.42.

### Test Example 25

### Synthesis of Hexyl Acrylate

The same reaction as that in Test Example 3 was carried out, with the exception that Novozym (registered trademark) 435 prepared by immersing it in a hydrochloric acid aqueous solution (pH 4) at 70° C. for 16 hours, and then adjusting the water content percentage thereof to 5.0% by vacuum drying, was used as a catalyst. The specific activity of the lipase in the transesterification reaction was 187.8 mmol·h⁻¹·g⁻¹. In addition, index value 1 (absorption band I, half-value width) obtained by the aforementioned method was 61.1 cm⁻¹, index value 2 (absorption band I, absorption band area ratio—two normal distributions) was 0.49, index value 3 (absorption band I, absorption band area ratio—three normal distributions) was 1.38, index value 4 (absorption band I, absorption band area ratio—five normal distributions) was 0.37, index value 5 (absorption band I, absorption band area ratio—eight normal distributions) was 0.63, index value 6 (absorption band I, absorption band area ratio—eight normal distributions) was 1.31, index value 7 (absorption band II, half-value width) was 40.0 cm⁻¹, and index value 8 (absorption band II, absorption band area ratio—three normal distributions) was 1.67.

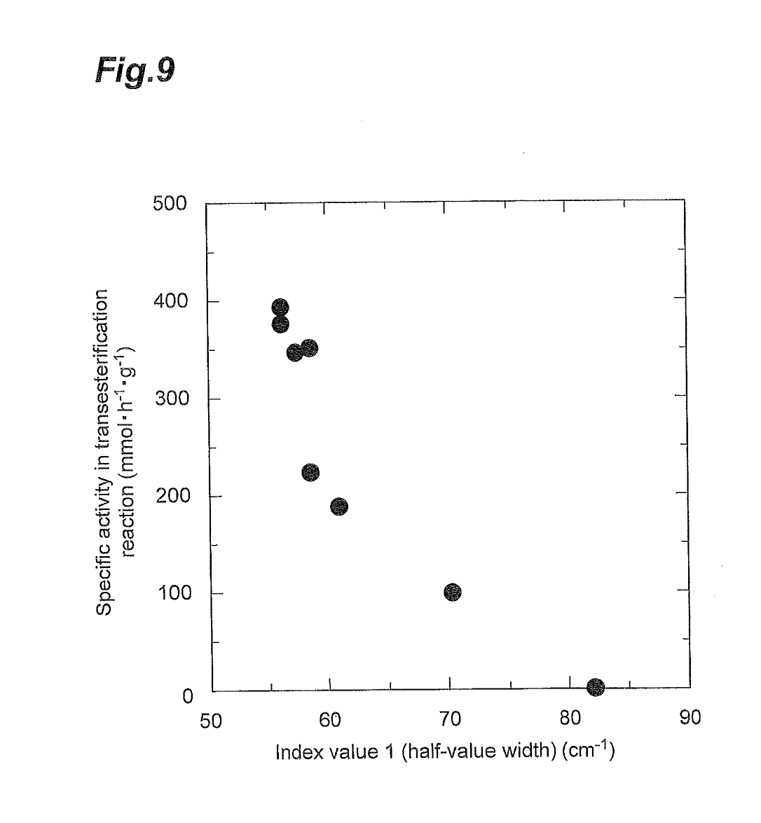

The results of the above described test examples are summarized in the following Table 1. In addition, FIGS. 9 to 16 each show a graph formed by plotting each index value (half-value width or absorption band area ratio), the specific activity of a lipase in a transesterification reaction, and the like.

FIG. 9 shows a graph formed by plotting the specific activity of a lipase in a transesterification reaction, with respect to index value 1 (absorption band I, half-value width) in Test Examples 17 to 25. A linearly approximatable negative correlation is observed.

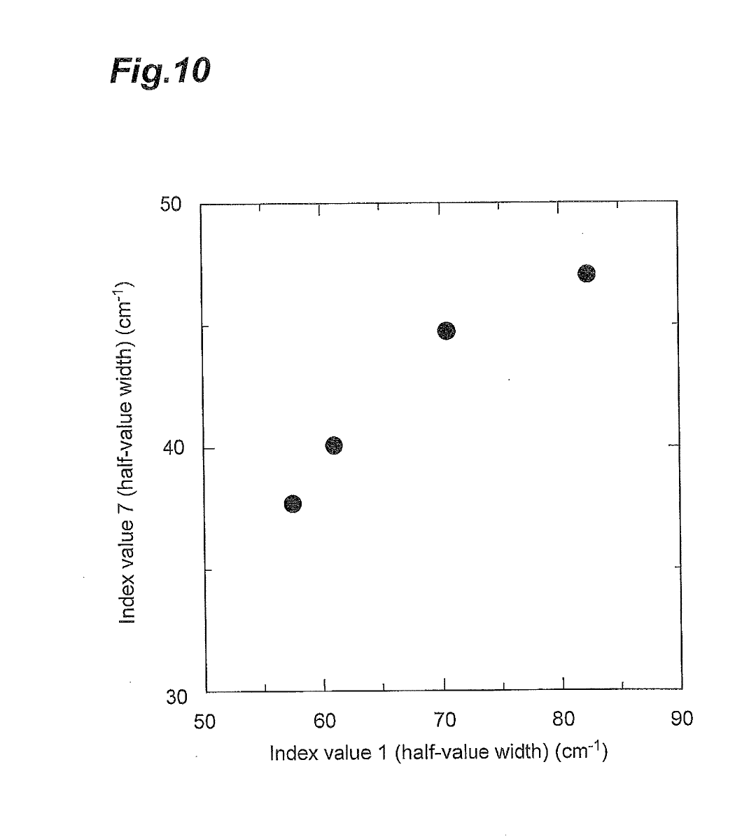

FIG. 10 shows a graph formed by plotting index value 7 (absorption band II, half-value width), with respect to index value 1 (absorption band I, half-value width) in Test Examples 17, 18, 22 and 25. A linearly approximatable positive correlation is observed.

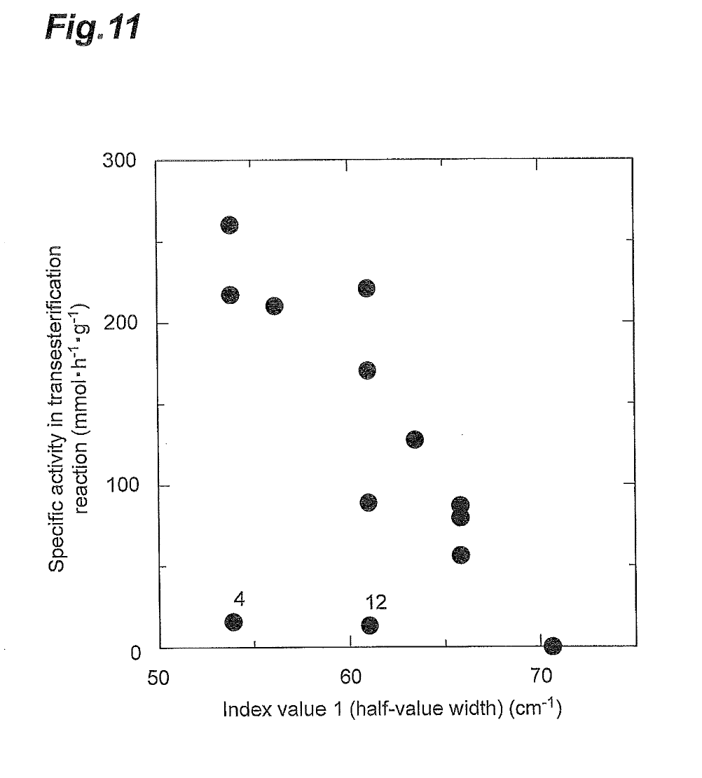

FIG. 11 shows a graph formed by plotting the specific activity of a lipase in a transesterification reaction, with respect to index value 1 (absorption band I, half-value width) in Test Examples 3 to 16. A linearly approximatable negative correlation is observed. It is to be noted that the results of Test Example 4 and Test Example 12 will be described later (in the figure, the number corresponding to each test example is assigned to the relevant plot).

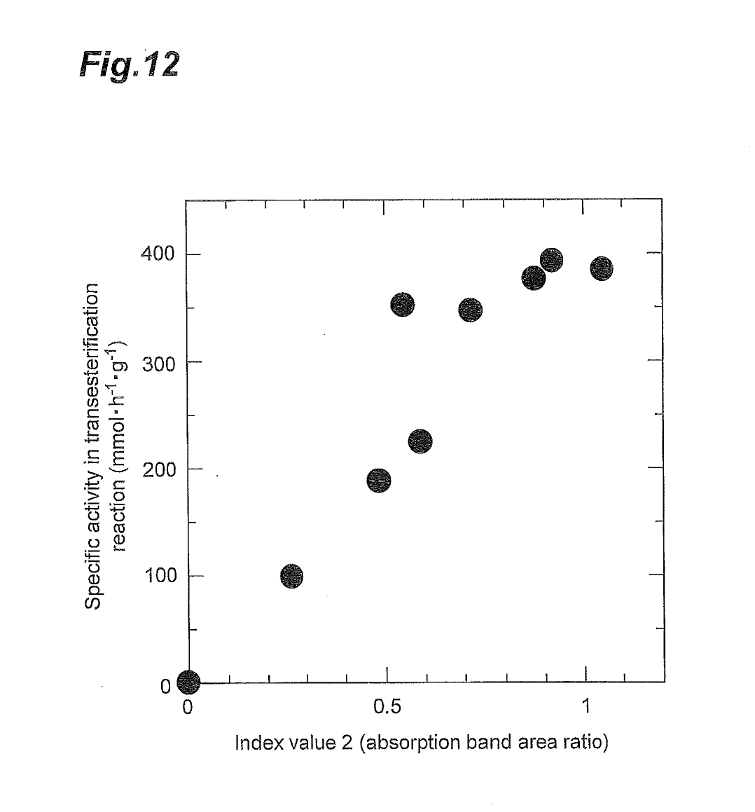

FIG. 12 shows a graph formed by plotting index value 2 (absorption band I, absorption band area ratio—two normal distributions) in Test Examples 17 to 25, and the specific activity of a lipase in a transesterification reaction. A linearly approximatable positive correlation is observed.

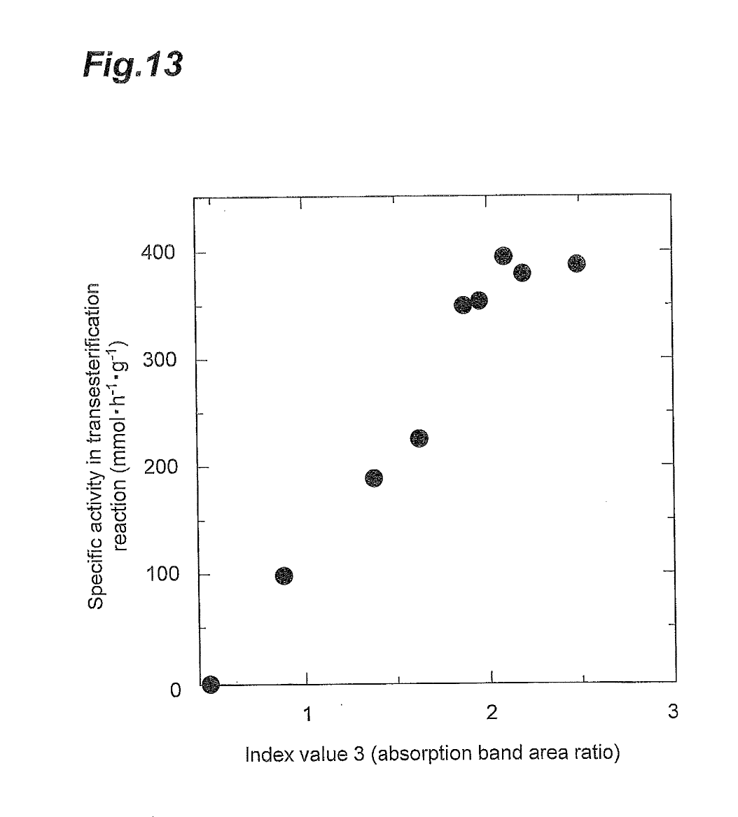

FIG. 13 shows a graph formed by plotting index value 3 (absorption band I, absorption band area ratio—three normal distributions) in Test Examples 17 to 25, and the specific activity of a lipase in a transesterification reaction. A linearly approximatable positive correlation is observed.

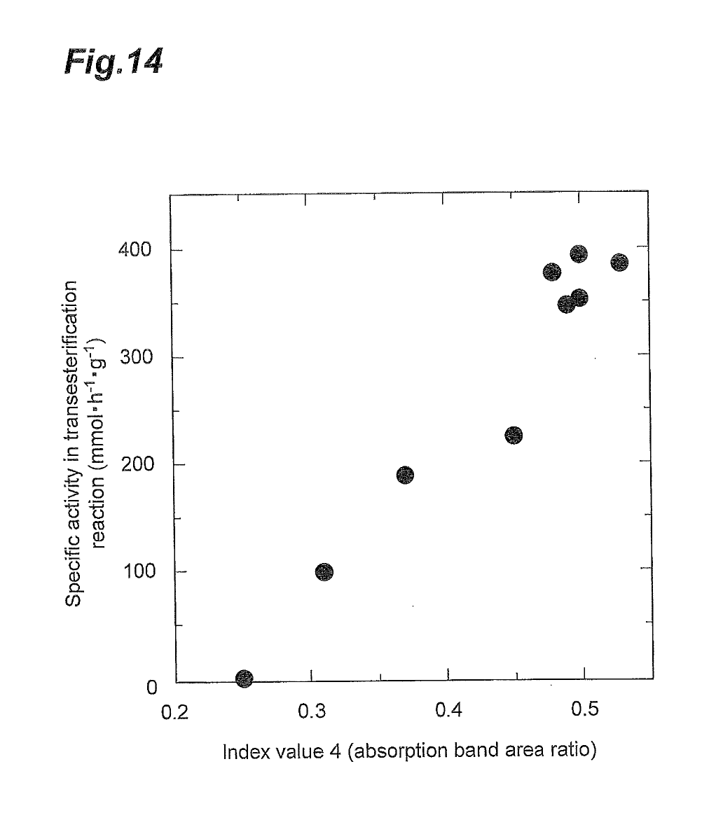

FIG. 14 shows a graph formed by plotting index value 4 (absorption band I, absorption band area ratio—five normal distributions) in Test Examples 17 to 25, and the specific activity of a lipase in a transesterification reaction. A linearly approximatable positive correlation is observed.

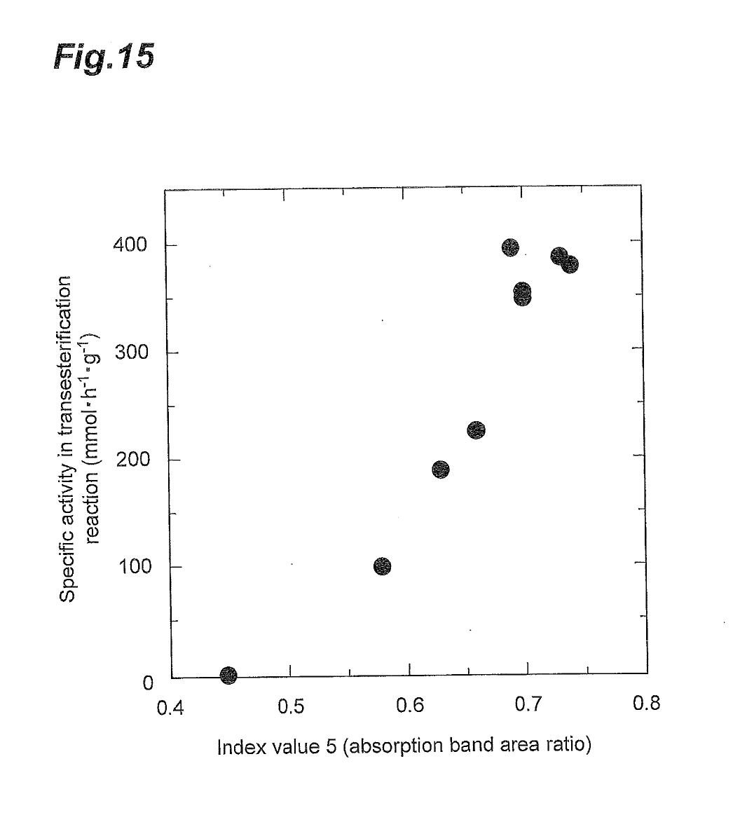

FIG. 15 shows a graph formed by plotting index value 5 (absorption band I, absorption band area ratio—eight normal distributions) in Test Examples 17 to 25, and the specific activity of a lipase in a transesterification reaction. A linearly approximatable positive correlation is observed.

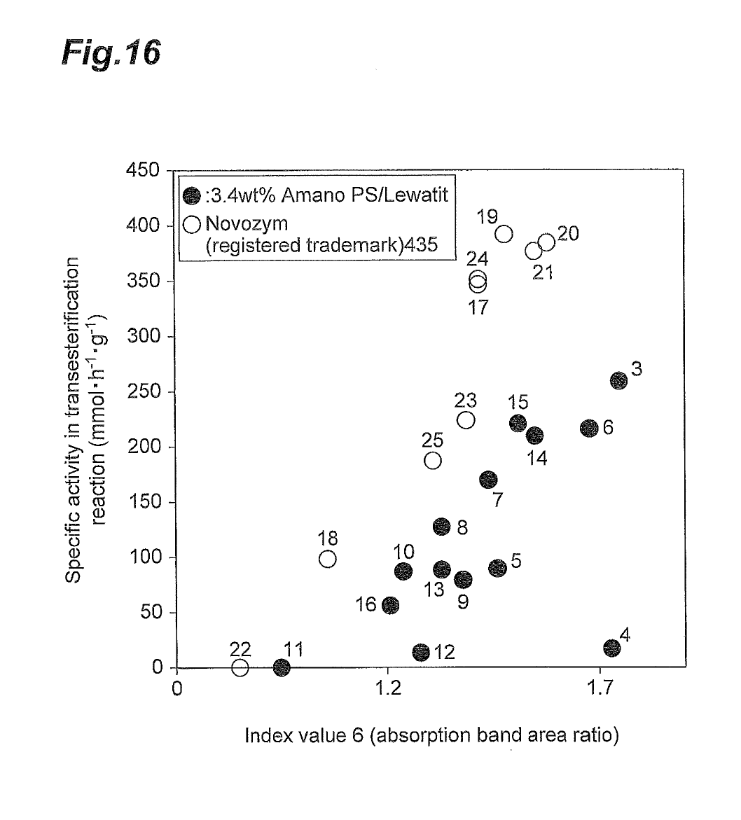

FIG. 16 shows a graph formed by plotting index value 6 (absorption band I, absorption band area ratio—eight normal distributions) in Test Examples 3 to 25, and the specific activity of a lipase in a transesterification reaction. A linearly approximatable positive correlation is observed.

TABLE 1

| Test | | | Water | | | | | | | | | Specific |
|---|---|---|---|---|---|---|---|---|---|---|---|---|
| Ex- | | | content | | | | | | | | | activity |
| am- | | | percentage | Index value | | | | | | | | (mmol · |
| ple | Immobilized lipase | Treatment | (%) | 1 | 2 | 3 | 4 | 5 | 6 | 7 | 8 | h⁻¹ · g⁻¹) |
| 3 | 3.4 wt % Amano PS/Lewatit | Not treated | 7.1 | 54.1 | — | — | — | — | 1.75 | — | — | 259.3 |
| 4 | 3.4 wt % Amano PS/Lewatit | Not treated | 1.0 | 54.1 | — | — | — | — | 1.73 | — | — | 16.5 |
| 5 | 3.4 wt % Amano PS/Lewatit | Methanol, 25° C., 2 hours | 71.0 | 61.1 | — | — | — | — | 1.46 | — | — | 89.8 |
| 6 | 3.4 wt % Amano PS/Lewatit | Toluene, 25° C., 2 hours | 50.0 | 54.1 | — | — | — | — | 1.68 | — | — | 216.1 |
| 7 | 3.4 wt % Amano PS/Lewatit | Acetone, 25° C., 2 hours | 52.4 | 61.1 | — | — | — | — | 1.44 | — | — | 169.7 |
| 8 | 3.4 wt % Amano PS/Lewatit | Acetonitrile, 25° C., 2 hours | 58.6 | 63.5 | — | — | — | — | 1.33 | — | — | 127.4 |
| 9 | 3.4 wt % Amano PS/Lewatit | 1-Butanol, 25° C., 2 hours | 65.5 | 65.8 | — | — | — | — | 1.38 | — | — | 79.7 |
| 10 | 3.4 wt % Amano PS/Lewatit | Ethanol, 25° C., 2 hours | 60.2 | 65.8 | — | — | — | — | 1.24 | — | — | 87.0 |
| 11 | 3.4 wt % Amano PS/Lewatit | 230° C., 1 hour | 40.0 | 70.5 | — | — | — | — | 0.95 | — | — | 0 |
| 12 | 3.4 wt % Amano PS/Lewatit | Used in reaction for 3 months | — | 61.1 | — | — | — | — | 1.28 | — | — | 13.6 |
| 13 | 3.4 wt % Amano PS/Lewatit | After used in reaction for | 46.1 | 61.1 | — | — | — | — | 1.33 | — | — | 88.2 |
| | | 3 months, in acetone with | | | | | | | | | | |
| | | water content percentage | | | | | | | | | | |
| | | of 5% at 6° C. overnight | | | | | | | | | | |
| 14 | 3.4 wt % Amano PS/Lewatit | Acetic acid aqueous | 25.0 | 56.4 | — | — | — | — | 1.55 | — | — | 209.6 |
| | | solution (pH 2), | | | | | | | | | | |
| | | 25° C., 2 hours | | | | | | | | | | |
| 15 | 3.4 wt % Amano PS/Lewatit | Hydrochloric acid aqueous | 12.0 | 61.1 | — | — | — | — | 1.51 | — | — | 220.5 |
| | | solution (pH 4), | | | | | | | | | | |
| | | 25° C., 16 hours | | | | | | | | | | |
| 16 | 3.4 wt % Amano PS/Lewatit | Hydrochloric acid aqueous | 33.0 | 65.8 | — | — | — | — | 1.21 | — | — | 56.1 |
| | | solution (pH 4), | | | | | | | | | | |
| | | 70° C., 16 hours | | | | | | | | | | |
| 17 | Novozym (registered | Not treated | 1.0 | 57.6 | 0.72 | 1.87 | 0.49 | 0.70 | 1.42 | 37.6 | 1.75 | 346.4 |
| | trademark) 435 | | | | | | | | | | | |
| 18 | Novozym (registered | Methanol, 25° C., 3 hours | 1.0 | 70.5 | 0.26 | 0.88 | 0.31 | 0.58 | 1.06 | 44.7 | 1.13 | 98.6 |
| | trademark) 435 | | | | | | | | | | | |
| 19 | Novozym (registered | Toluene, 25° C., 3 hours | 1.0 | 56.4 | 0.92 | 2.10 | 0.50 | 0.69 | 1.48 | — | — | 392.6 |
| | trademark) 435 | | | | | | | | | | | |
| 20 | Novozym (registered | Acetone, 25° C., 3 hours | 1.0 | 56.4 | 1.05 | 2.50 | 0.53 | 0.73 | 1.58 | — | — | 384.4 |
| | trademark) 435 | | | | | | | | | | | |
| 21 | Novozym (registered | Acetonitrile, 25° C., 3 hours | 1.0 | 56.4 | 0.88 | 2.20 | 0.48 | 0.74 | 1.55 | — | — | 375.9 |
| | trademark) 435 | | | | | | | | | | | |
| 22 | Novozym (registered | 230° C., 1 hour | 0.1 | 82.3 | 0 | 0.47 | 0.25 | 0.45 | 0.85 | 47.0 | 0.58 | 0 |
| | trademark) 435 | | | | | | | | | | | |
| 23 | Novozym (registered | Acetic acid aqueous | 9.0 | 58.8 | 0.59 | 1.63 | 0.45 | 0.66 | 1.39 | — | — | 224.2 |
| | trademark) 435 | solution (pH 2), | | | | | | | | | | |
| | | 25° C., 2 hours | | | | | | | | | | |
| 24 | Novozym (registered | Hydrochloric acid aqueous | 3.0 | 58.8 | 0.55 | 1.97 | 0.50 | 0.70 | 1.42 | — | — | 351.6 |
| | trademark) 435 | solution (pH 4), | | | | | | | | | | |
| | | 25° C., 16 hours | | | | | | | | | | |
| 25 | Novozym (registered | Hydrochloric acid aqueous | 5.0 | 61.1 | 0.49 | 1.38 | 0.37 | 0.63 | 1.31 | 40.0 | 1.13 | 187.8 |
| | trademark) 435 | solution (pH 4), | | | | | | | | | | |
| | | 70° C., 16 hours | | | | | | | | | | |

As is clear from Table 1 and FIGS. 9 to 16, with regard to the immobilized lipase having a sufficient water content percentage (which probably has not undergone reversible denaturation), a linearly approximatable correlation is observed between the index value (half-value width or absorption band area ratio) and the catalytic activity (the specific activity of the lipase in the transesterification reaction). Because of this correlation, it can be evaluated that an immobilized lipase, in which the half-value width is a predetermined value or less or the absorption band area ratio is a predetermined value or more (e.g., in the case of index value 6, it is 1.2 or more), has a sufficient catalytic activity or a sufficient potential catalytic activity.

Moreover, from a comparison between Test Example 12 and Test Example 13, it can be understood that a catalytic activity that corresponds to the absorption band area ratio can be reactivated by dissolving reversible denaturation by performing a treatment of using a hydrous organic solvent. That is to say, the immobilized lipase of Test Example 13 is prepared by treating the immobilized lipase of Test Example 12 with acetone having a water content percentage of 5%. As is clear from the results shown in Table 1, the catalytic activity was reactivated by the aforementioned treatment, and as shown in FIG. 16 for example, it then entered the linear approximation curve of 3.4 wt % Amano PS/Lewatit (not shown in the figure). The immobilized lipases of Test Example 13 and Test Example 12 have almost the same index value (e.g., the half-value width of index value 1 is 61.1 cm⁻¹ in both of the test examples, and the absorption band area ratios of index value 6 are 1.33 and 1.28 in the aforementioned test examples, respectively).

Furthermore, also from a comparison between Test Example 3 and Test Example 4, it can be understood that a catalytic activity that corresponds to the absorption band area ratio can be reactivated by dissolving reversible denaturation. In the case of the immobilized lipases of Test Example 3 and Test Example 4, only their water content percentages are different from each other (wherein the immobilized lipase of Test Example 4 having a low water content percentage is considered to undergo reversible denaturation), and Test Example 3 and Test Example 4 have almost the same index value.

In the conventional method, which comprises actually carrying out a catalytic reaction, measuring a catalytic activity, and evaluating the catalytic activity of an immobilized lipase, the immobilized lipases of, for example, Test Example 12 and Test Example 4 are discarded as defective products, although the catalytic activity of these lipases can be reactivated. On the other hand, according to the method of the present invention, since the immobilized lipases of Test Example 12 and Test Example 4 have a half-value width that is a predetermined value or less, or an absorption band area ratio that is a predetermined value or more, they can be selected as immobilized lipases capable of reactivating their catalytic activity.

[2. Evaluation of the Catalytic Activity of an Immobilized Peroxidase]

<Method of Quantifying Oxide of o-phenylenediamine>

The oxide of o-phenylenediamine was quantified by measuring a UV-vis spectrum with an ultraviolet visible spectrophotometer. Measurement conditions for the ultraviolet visible spectrophotometer are as follows.
Ultraviolet visible spectrophotometer (UV2450, manufactured by Shimadzu Corporation)
Peak wavelength: 420 nm

<Method of Calculating a Specific Activity in an Oxidation Reaction>

Using the peak intensity (absorption intensity at 420 nm) of a UV-vis spectrum derived from the oxide of o-phenylenediamine measured by the above described method, the specific activity of an immobilized peroxidase in an oxidation reaction was calculated according to the following formula. It is to be noted that the weight of oxidase means the weight of peroxidase that was obtained relative to BSA according to a BCA method.

Specific activity in oxidation reaction (g⁻¹·h⁻¹)=(Absorption intensity at 420 nm)/(Oxidase weight (g)×reaction time (h)) [Expression 2]

<Method of Measuring a Water Content Percentage>

The water content percentage was measured by the same method as in the case of an immobilized lipase.

### Test Example 26

### Calculation of Index Values

The index value was calculated in the same manner as that in Test Example 1, with the exception that baseline correction was carried out, so that the infrared absorption intensity at 1800 and 1545 cm⁻¹ could be 0.

### Test Example 27

### Preparation of an Immobilized Peroxidase (2.6 wt % HRP/MCFs)

400 g of 20% HCl aqueous solution and 200 g of purified water were added to 33.2 g of Pluronic P-123, and then, 23.5 g of mesitylene was added thereto. Thereafter, the mixture was immersed in a water bath that had been heated to 40° C., so that Pluronic P-123 was fully dissolved in the solution. Thereafter, 70.0 g of tetraethoxysilane was added to the reaction solution, the obtained solution was then stirred for 5 minutes, and it was then left at rest at 30° C. for 20 hours. Thereafter, an ammonium fluoride aqueous solution (0.38 g/40 g-H₂O) was added to the obtained white slurry, and the obtained mixture was then matured at 100° C. for 24 hours. Thereafter, the resultant was washed with 500 mL of a mixed solution of purified water and ethanol; and was then filtrated, and it was then dried in an oven at 100° C. overnight. The temperature of the obtained white powder was increased to 500° C. in the atmosphere over 5 hours, and the white powder was then retained at the same temperature for 5 hours. The thus obtained white powder (Siliceous Mesocellular Foams, MCFs) is referred to as a “silica carrier” or “MCFs.” The specific surface area and mean small pore diameter of the silica carrier obtained by nitrogen adsorption desorption measurement were 597 m²·g⁻¹ and 24.4 nm, respectively.

82 mg of horseradish peroxidase (HRP, 100 units·mg⁻¹, manufactured by Wako Pure Chemical Industries, Ltd.) was dissolved in 8.1 g of 50 mM sodium phosphate buffer solution (pH 7) to prepare an immobilization stock solution. 7.0 g of the immobilization stock solution was weighed, and 0.35 g of carrier was then added thereto. The obtained slurry was stirred at 7° C. for 24 hours. The amount of a peroxidase immobilized was calculated by quantifying the concentration of a protein in the immobilization stock solution and the concentration of a protein in a supernatant solution obtained by centrifugation of the slurry according to a BCA method, and then calculating the immobilized amount from a difference between the two concentrations. As a result, the amount of the peroxidase immobilized was 2.6 wt %. This immobilized peroxidase is referred to as an “immobilized peroxidase” or “2.6 wt % HRP/MCFs.”

### Test Example 28

### Oxidation Reaction of o-phenylenediamine

2.5 mL of toluene solution of 0.1 M o-phenylenediamine, 0.6 mL of decane solution of 1.1 M t-butyl hydroperoxide, and an immobilized peroxidase having a water content percentage of 34.9% used as a catalyst were added to a glass test tube having an internal volume of 20 mL that was equipped with a stirrer and a temperature-control device, and the obtained mixture was then reacted at 35° C., while being stirred. One hour later, the reaction solution was removed, and the specific activity of the peroxidase in the oxidation reaction was then calculated according to the aforementioned method. The specific activity of the peroxidase in the oxidation reaction was 3899.9 h⁻¹·g⁻¹. In addition, index value 7 (absorption band II, half-value width) obtained by the aforementioned method was 65.7 cm⁻¹, and index value 8 (absorption band II, absorption band area ratio—three normal distributions) was 0.70.

### Test Example 29

### Oxidation Reaction of o-phenylenediamine

The same reaction as that in Test Example 28 was carried out, with the exception that an immobilized peroxidase prepared by immersing it in methanol at 25° C. for 3 hours, and then washing it with purified water to adjust the water content percentage thereof to 22.7%, was used as a catalyst. The specific activity of the peroxidase in the oxidation reaction was 1016.0 h⁻¹·g⁻¹. In addition, index value 7 (absorption band II, half-value width) obtained by the aforementioned method was 75.1 cm⁻¹, and index value 8 (absorption band II, absorption band area ratio—three normal distributions) was 0.40.

### Test Example 30

### Oxidation Reaction of o-phenylenediamine

The same reaction as that in Test Example 28 was carried out, with the exception that an immobilized peroxidase prepared by immersing it in toluene at 110° C. for 3 hours, and then washing it with purified water to adjust the water content percentage thereof to 28.5%, was used as a catalyst. The specific activity of the peroxidase in the oxidation reaction was 2841.5 h⁻¹·g⁻¹. In addition, index value 7 (absorption band II, half-value width) obtained by the aforementioned method was 71.1 cm⁻¹, and index value 8 (absorption band II, absorption band area ratio—three normal distributions) was 0.62.

### Test Example 31

### Oxidation Reaction of o-phenylenediamine

The same reaction as that in Test Example 28 was carried out, with the exception that an immobilized peroxidase prepared by immersing it in an HCl aqueous solution (pH 3.5) at 60° C. for 3 hours, and then washing it with purified water to adjust the water content percentage thereof to 25.3%, was used as a catalyst. The specific activity of the peroxidase in the oxidation reaction was 2374.8 h⁻¹·g⁻¹. In addition, index value 7 (absorption band II, half-value width) obtained by the aforementioned method was 70.5 cm⁻¹, and index value 8 (absorption band II, absorption band area ratio—three normal distributions) was 0.58.

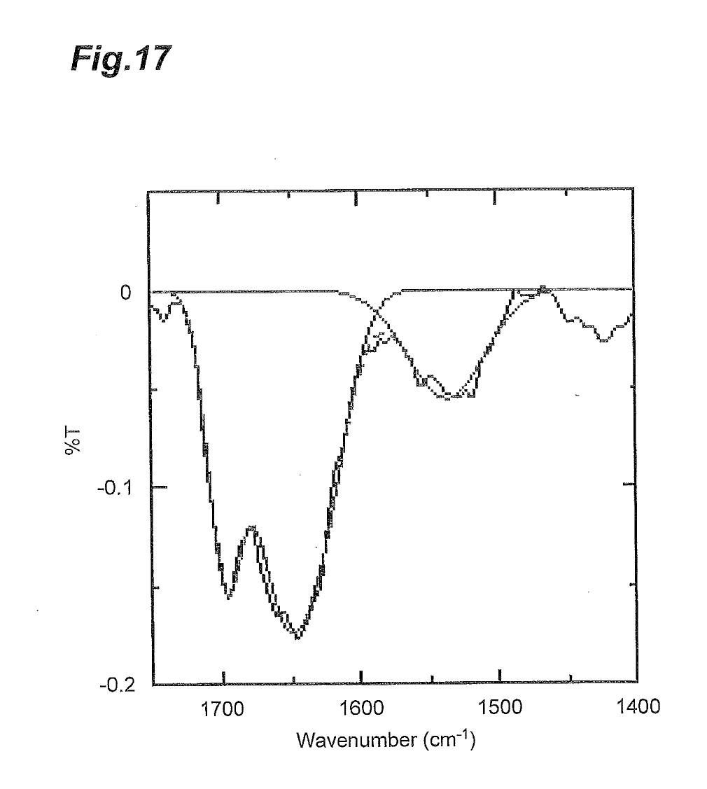

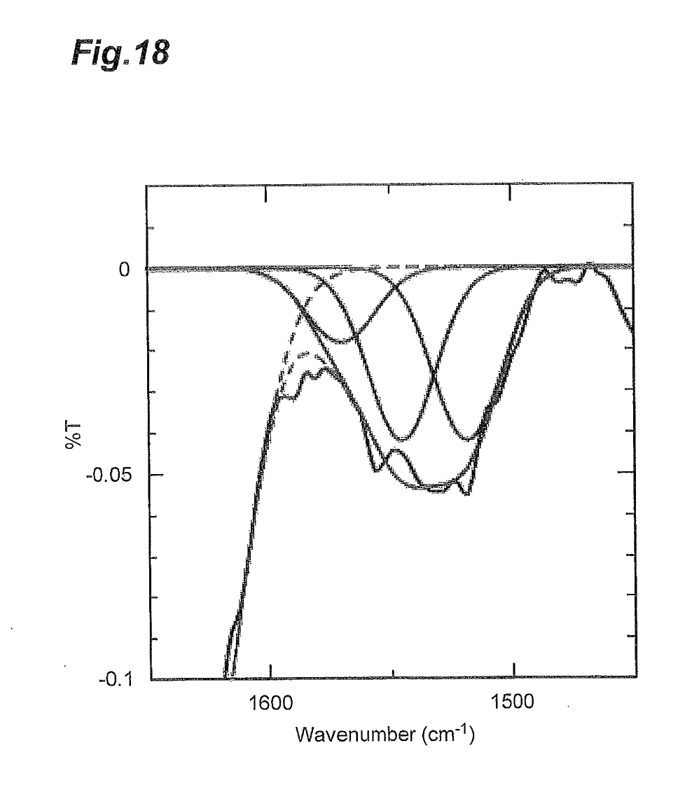

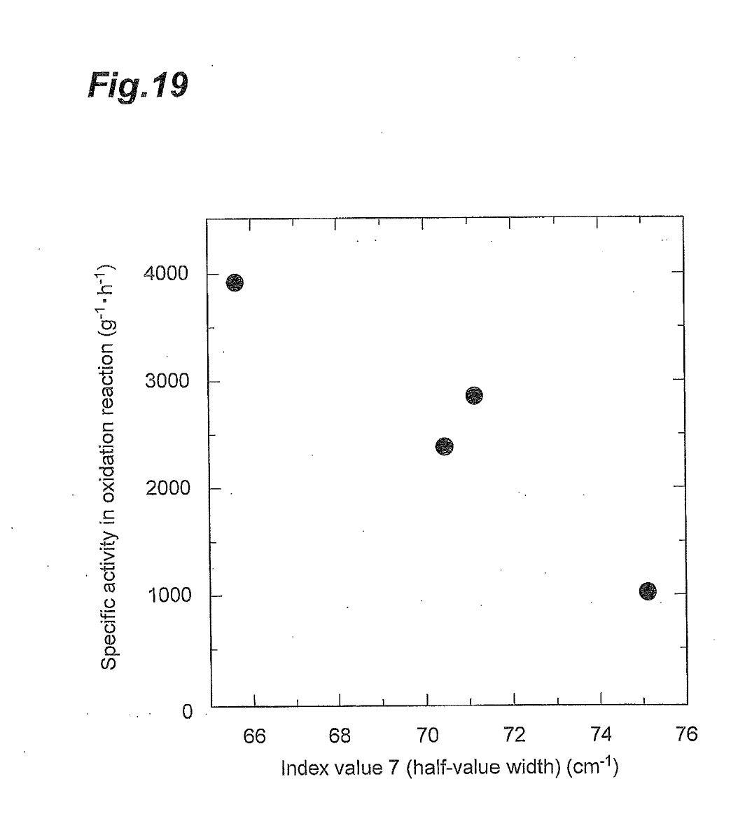

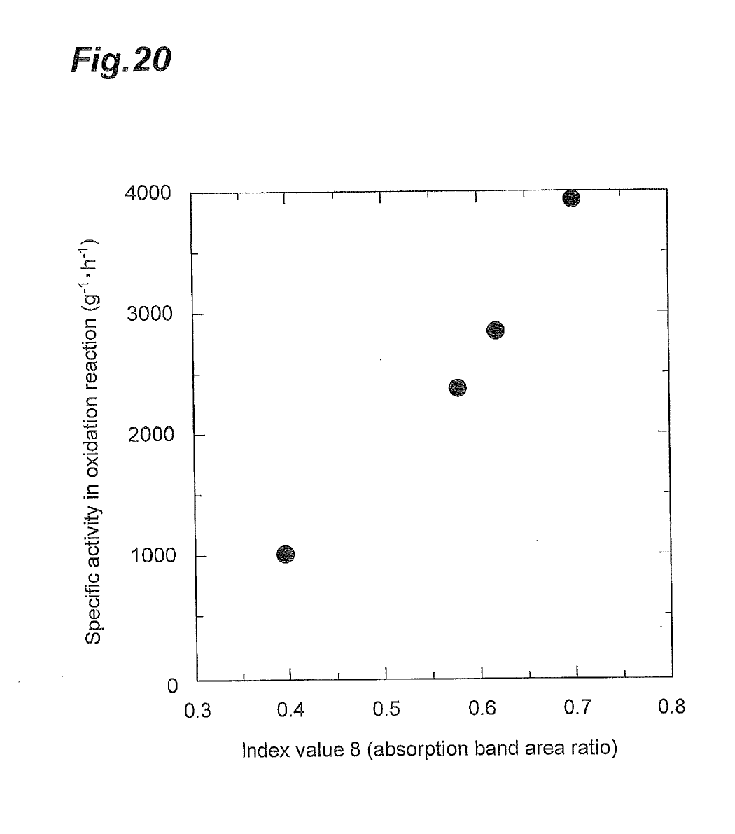

The results of Test Examples 28 to 31 are summarized in the following Table 2. In addition, FIGS. 17 and 18 show an example of approximating an infrared absorption band derived from a peroxidase by a single normal distribution, and an example of subjecting such an infrared absorption band to waveform separation, respectively. FIGS. 19 and 20 each show a graph obtained by plotting each index value (half-value width or absorption band area ratio) and the specific activity of a peroxidase in an oxidation reaction.

FIG. 17 shows the FT-IR spectrum of the immobilized peroxidase used as a catalyst in Test Example 28, and an example of approximating absorption band II derived from a peroxidase by a single normal distribution. FIG. 18 shows the FT-IR spectrum of the immobilized peroxidase used as a catalyst in Test Example 28, and an example of subjecting absorption band II derived from a peroxidase to waveform separation to obtain three normal distributions.

FIG. 19 shows a graph obtained by plotting the specific activity of a peroxidase in an oxidation reaction, with respect to index value 7 (absorption band II, half-value width) in Test Examples 28 to 31. A linearly approximatable negative correlation is observed.

FIG. 20 shows a graph obtained by plotting the specific activity of a peroxidase in an oxidation reaction, with respect to index value 8 (absorption band II, absorption band area ratio—three normal distributions) in Test Examples 28 to 31. A linearly approximatable positive correlation is observed.

TABLE 2

| | | | | | | Specific activity in |
|---|---|---|---|---|---|---|
| Test | Immobilized | | Water content | Index | Index | oxidation reaction |
| Example | peroxidase | Treatment | percentage (%) | value 7 | value 8 | (h⁻¹ · g⁻¹) |
| 28 | 2.6 wt % HRP/MCFs | Not treated | 34.9 | 65.7 | 0.70 | 3899.9 |
| 29 | 2.6 wt % HRP/MCFs | Methanol, 25° C., 3 hours | 22.7 | 75.1 | 0.40 | 1016.0 |
| 30 | 2.6 wt % HRP/MCFs | Toluene, 110° C., 3 hours | 28.5 | 71.1 | 0.62 | 2841.5 |
| 31 | 2.6 wt % HRP/MCFs | HCl aqueous solution (pH | 25.3 | 70.5 | 0.58 | 2374.8 |
| | | 3.5), 60° C., 3 hours | | | | |

As is clear from Table 2 and FIGS. 19 and 20, with regard to the immobilized peroxidase having a sufficient water content percentage (which probably has not undergone reversible denaturation), a linearly approximatable correlation is observed between the index value (half-value width or absorption band area ratio) and the catalytic activity (the specific activity in the oxidation reaction). Because of this correlation, it can be evaluated that an immobilized peroxidase, in which the half-value width is a predetermined value or less, or the absorption band area ratio is a predetermined value or more, has a sufficient catalytic activity or a sufficient potential catalytic activity.

[3. Evaluation of Antibody Titer]

### <Method of Measuring Antibody Titer>

The titer of an antibody (IgG) was measured by a direct adsorption method ELISA (Enzyme-Linked Immunosorbent Assay).

A PBS (Phosphate Buffered Saline: 10 mM phosphoric acid (pH 7.4), 0.14 M NaCl, 0.0027 M KCl) buffer was added to 50 μg of antigen (chicken IgG) to prepare a 1000 ng/mL antigen solution. From the 1000 ng/mL antigen solution, 1000, 500, 250, 125, 63, 31 and 16 ng/mL antigen solutions were prepared by two-fold dilution of using PBS. 50 μL of each antigen solution was added to each well of a 96-well plate, and it was then left for 5 minutes, so that the antigen was adsorbed on each well. Thereafter, each well was washed with PBS twice, so as to obtain a 96-well plate on which the antigen was adsorbed.

The antibody that was to be a target of the titer measurement was dissolved in and diluted with PBS containing 0.05% Tween 20, to prepare a 1 μg/mL antibody solution (which is referred to as a primary antibody solution). 50 μL of the primary antibody solution was added to each well of the 96-well plate on which the antigen had been adsorbed, and it was then left for 5 minutes, so that the antibody was allowed to bind to the antigen. Each well was washed with PBS containing 0.05% Tween 20 twice.

50 μg of a horseradish peroxidase (HRP)-labeled goat anti-rabbit antibody was dissolved in and diluted with PBS containing 0.05% Tween 20, to prepare a 1 μg/mL antibody solution (which is referred to as a secondary antibody solution). 50 μL of the secondary antibody solution was added to each well of the 96-well plate to which the primary antibody had been allowed to bind, and it was then left for 5 minutes, so that secondary antibody was allowed to bind to the primary antibody. Each well was washed with PBS containing 0.05% Tween 20 twice.

50 μL of enzyme substrate solution (3,3′,5,5′-tetramethylbenzidine (TMB), manufactured by Bio-Rad Laboratories, Inc.) was added to each well of the 96-well plate to which the secondary antibody had been allowed to bind, and it was then left at a room temperature for 5 minutes, and thereafter, the absorbance at 655 nm was measured by using a microplate reader (manufactured by Tecan Trading AG).

### Test Example 32

### Calculation of Index Values

The index value was calculated in the same manner as that in Test Example 1, with the exception that baseline correction was carried out, so that the infrared absorption intensity at 1720 and 1490 cm⁻¹ could be 0.

### Test Example 33

### Measurement of Antibody Titer

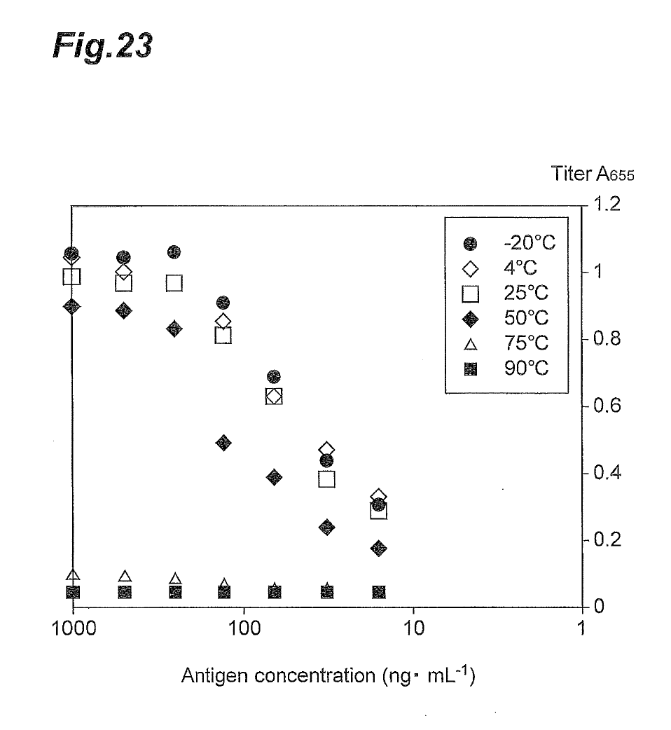

45 mg of polyethylene glycol (PEG; average molecular weight: 20000) was added to 0.5 mL of anti-chicken IgG rabbit antibody (manufactured by Rockland Immunochemicals Inc.) solution (10.0 mg/mL), and it was then dissolved in 5 mL of 10 mM phosphate buffer solution (pH 7.0) to prepare an antibody solution. Thereafter, 0.5 mL of the antibody solution was fractionated in a test tube, and it was immediately freeze-dried at −20° C. After completion of the freeze-drying, the antibody was used in the measurement of a titer and the measurement of an FT-IR spectrum according to a microscopic ATR method. The results obtained by measuring the titer of the antibody are shown in Table 3 and FIG. 23 (in FIG. 23, Test Example 33 corresponds to the plot “−20° C.”). In addition, index value 1 (absorption band I, half-value width) obtained by the aforementioned method was 57.0 cm⁻¹, index value 6 (absorption band I, absorption band area ratio—eight normal distributions) was 1.09, and index value 8 (absorption band II, absorption band area ratio—three normal distributions) was 0.97.

### Test Example 34

### Measurement of Antibody Titer

The measurement was carried out in the same manner as that in Test Example 33, with the exception that 0.5 mL of the antibody solution was fractionated in a test tube, it was then left at rest at 4° C. for 24 hours, and it was then freeze-dried at −20° C. The results obtained by measuring the titer of the antibody are shown in Table 3 and FIG. 23 (in FIG. 23, Test Example 34 corresponds to the plot “4° C.”). In addition, index value 1 (absorption band I, half-value width) obtained by the aforementioned method was 57.6 cm⁻¹, index value 6 (absorption band I, absorption band area ratio—eight normal distributions) was 1.03, and index value 8 (absorption band II, absorption band area ratio—three normal distributions) was 0.92.

### Test Example 35

### Measurement of Antibody Titer

The measurement was carried out in the same manner as that in Test Example 33, with the exception that 0.5 mL of the antibody solution was fractionated in a test tube, it was then left at rest at 25° C. for 24 hours, and it was then freeze-dried at −20° C. The results obtained by measuring the titer of the antibody are shown in Table 3 and FIG. 23 (in FIG. 23, Test Example 35 corresponds to the plot “25° C.”). In addition, index value 1 (absorption band I, half-value width) obtained by the aforementioned method was 57.7 cm⁻¹, index value 6 (absorption band I, absorption band area ratio—eight normal distributions) was 1.00, and index value 8 (absorption band II, absorption band area ratio—three normal distributions) was 0.94.

### Test Example 36

### Measurement of Antibody Titer

The measurement was carried out in the same manner as that in Test Example 33, with the exception that 0.5 mL of the antibody solution was fractionated in a test tube, it was then left at rest at 50° C. for 24 hours, and it was then freeze-dried at −20° C. The results obtained by measuring the titer of the antibody are shown in Table 3 and FIG. 23 (in FIG. 23, Test Example 36 corresponds to the plot “50° C.”). In addition, index value 1 (absorption band I, half-value width) obtained by the aforementioned method was 58.4 cm⁻¹, index value 6 (absorption band I, absorption band area ratio—eight normal distributions) was 1.02, and index value 8 (absorption band II, absorption band area ratio—three normal distributions) was 0.93.

### Test Example 37

### Measurement of Antibody Titer

The measurement was carried out in the same manner as that in Test Example 33, with the exception that 0.5 mL of the antibody solution was fractionated in a test tube, it was then left at rest at 75° C. for 24 hours, and it was then freeze-dried at −20° C. The results obtained by measuring the titer of the antibody are shown in Table 3 and FIG. 23 (in FIG. 23, Test Example 37 corresponds to the plot “75° C.”). In addition, index value 1 (absorption band I, half-value width) obtained by the aforementioned method was 66.0 cm⁻¹, index value 6 (absorption band I, absorption band area ratio—eight normal distributions) was 0.96, and index value 8 (absorption band II, absorption band area ratio—three normal distributions) was 0.80.

### Test Example 38

### Measurement of Antibody Titer

The measurement was carried out in the same manner as that in Test Example 33, with the exception that 0.5 mL of the antibody solution was fractionated in a test tube, it was then left at rest at 90° C. for 24 hours, and it was then freeze-dried at −20° C. The results obtained by measuring the titer of the antibody are shown in Table 3 and FIG. 23 (in FIG. 23, Test Example 38 corresponds to the plot “90° C.”). In addition, index value 1 (absorption band I, half-value width) obtained by the aforementioned method was 70.6 cm⁻¹, index value 6 (absorption band I, absorption band area ratio—eight normal distributions) was 0.92, and index value 8 (absorption band II, absorption band area ratio—three normal distributions) was 0.79.

### Test Example 39

### Measurement of Antibody Titer

The measurement was carried out in the same manner as that in Test Example 33, with the exception that 0.5 mL of the antibody solution was fractionated in a test tube, acetone was then added thereto resulting in 10% v/v, the mixture was then left at rest at 25° C. for 24 hours, and nitrogen was then purged for 10 minutes to sublimate the acetone, and the resultant was then freeze-dried at −20° C. The results obtained by measuring the titer of the antibody are shown in Table 3. In addition, index value 1 (absorption band I, half-value width) obtained by the aforementioned method was 56.9 cm⁻¹, index value 6 (absorption band I, absorption band area ratio—eight normal distributions) was 0.98, and index value 8 (absorption band II, absorption band area ratio—three normal distributions) was 0.95.

### Test Example 40

### Measurement of Antibody Titer

The measurement was carried out in the same manner as that in Test Example 33, with the exception that 0.5 mL of the antibody solution was fractionated in a test tube, acetone was then added thereto resulting in 20% v/v, the mixture was then left at rest at 25° C. for 24 hours, and nitrogen was then purged for 10 minutes to sublimate the acetone, and the resultant was then freeze-dried at −20° C. The results obtained by measuring the titer of the antibody are shown in Table 3. In addition, index value 1 (absorption band I, half-value width) obtained by the aforementioned method was 56.7 cm^{31 1}, index value 6 (absorption band I, absorption band area ratio—eight normal distributions) was 1.01, and index value 8 (absorption band II, absorption band area ratio—three normal distributions) was 0.97.

### Test Example 41

### Measurement of Antibody Titer

The measurement was carried out in the same manner as that in Test Example 33, with the exception that 0.5 mL of the antibody solution was fractionated in a test tube, acetone was then added thereto resulting in 40% v/v, the mixture was then left at rest at 25° C. for 24 hours, and nitrogen was then purged for 10 minutes to sublimate the acetone, and the resultant was then freeze-dried at −20° C. The results obtained by measuring the titer of the antibody are shown in Table 3. In addition, index value 1 (absorption band I, half-value width) obtained by the aforementioned method was 57.0 cm⁻¹, index value 6 (absorption band I, absorption band area ratio—eight normal distributions) was 1.00, and index value 8 (absorption band II, absorption band area ratio—three normal distributions) was 0.96.

### Test Example 42

### Measurement of Antibody Titer

The measurement was carried out in the same manner as that in Test Example 33, with the exception that 0.5 mL of the antibody solution was fractionated in a test tube, acetone was then added thereto resulting in 50% v/v, the mixture was then left at rest at 25° C. for 24 hours, and nitrogen was then purged for 10 minutes to sublimate the acetone, and the resultant was then freeze-dried at −20° C. The results obtained by measuring the titer of the antibody are shown in Table 3. In addition, index value 1 (absorption band I, half-value width) obtained by the aforementioned method was 57.8 cm⁻¹, index value 6 (absorption band I, absorption band area ratio—eight normal distributions) was 1.00, and index value 8 (absorption band II, absorption band area ratio—three normal distributions) was 0.93.

### Test Example 43

### Calculation of Index Value

45 mg of polyethylene glycol (PEG; average molecular weight: 20000) was added to 0.5 mL of anti-chicken egg-white lysozyme antibody (Anti White Lysozyme, manufactured by COSMO BIO Co., Ltd.) solution (10.0 mg/ mL), and it was then dissolved in 5 mL of 10 mL phosphate buffer solution (pH 7.0) to prepare an antibody solution. Thereafter, 0.5 mL of the antibody solution was fractionated in a test tube, and it was immediately freeze-dried at −20° C. After completion of the freeze-drying, the antibody was used in the measurement of an FT-IR spectrum according to a microscopic ATR method. Index value 1 (absorption band I, half-value width) obtained by the aforementioned method was 57.1 cm⁻¹.

### Test Example 44

### Calculation of Index Value

The measurement was carried out in the same manner as that in Test Example 43, with the exception that 0.5 mL of the antibody solution was fractionated in a test tube, it was then left at rest at 4° C. for 24 hours, and it was then freeze-dried at −20° C. Index value 1 (absorption band I, half-value width) obtained by the aforementioned method was 57.4 cm⁻¹.

### Test Example 45

### Calculation of Index Value

The measurement was carried out in the same manner as that in Test Example 43, with the exception that 0.5 mL of the antibody solution was fractionated in a test tube, it was then left at rest at 25° C. for 24 hours, and it was then freeze-dried at −20° C. Index value 1 (absorption band I, half-value width) obtained by the aforementioned method was 58.6 cm⁻¹.

### Test Example 46

### Calculation of Index Value

The measurement was carried out in the same manner as that in Test Example 43, with the exception that 0.5 mL of the antibody solution was fractionated in a test tube, it was then left at rest at 50° C. for 24 hours, and it was then freeze-dried at −20° C. Index value 1 (absorption band I, half-value width) obtained by the aforementioned method was 62.5 cm⁻¹.

### Test Example 47

### Calculation of Index Value

The measurement was carried out in the same manner as that in Test Example 43, with the exception that 0.5 mL of the antibody solution was fractionated in a test tube, it was then left at rest at 75° C. for 24 hours, and it was then freeze-dried at −20° C. Index value 1 (absorption band I, half-value width) obtained by the aforementioned method was 72.5 cm⁻¹.

### Test Example 48

### Calculation of Index Value

The measurement was carried out in the same manner as that in Test Example 43, with the exception that 0.5 mL of the antibody solution was fractionated in a test tube, it was then left at rest at 90° C. for 24 hours, and it was then freeze-dried at −20° C. Index value 1 (absorption band I, half-value width) obtained by the aforementioned method was 94.0 cm⁻¹.

### Test Example 49

### Calculation of Index Value

The measurement was carried out in the same manner as that in Test Example 43, with the exception that 0.5 mL of the antibody solution was fractionated in a test tube, acetone was then added thereto resulting in 10% v/v, the mixture was then left at rest at 25° C. for 24 hours, and nitrogen was then purged for 10 minutes to sublimate the acetone, and the resultant was then freeze-dried at −20° C. Index value 1 (absorption band I, half-value width) obtained by the aforementioned method was 58.4 cm⁻¹.

### Test Example 50

### Calculation of Index Value

The measurement was carried out in the same manner as that in Test Example 43, with the exception that 0.5 mL of the antibody solution was fractionated in a test tube, acetone was then added thereto resulting in 20% v/v, the mixture was then left at rest at 25° C. for 24 hours, and nitrogen was then purged for 10 minutes to sublimate the acetone, and the resultant was then freeze-dried at −20° C. Index value 1 (absorption band I, half-value width) obtained by the aforementioned method was 59.0 cm⁻¹.

### Test Example 51

### Calculation of Index Value

The measurement was carried out in the same manner as that in Test Example 43, with the exception that 0.5 mL of the antibody solution was fractionated in a test tube, acetone was then added thereto resulting in 40% v/v, the mixture was then left at rest at 25° C. for 24 hours, and nitrogen was then purged for 10 minutes to sublimate the acetone, and the resultant was then freeze-dried at −20° C. Index value 1 (absorption band I, half-value width) obtained by the aforementioned method was 59.9 cm⁻¹.

### Test Example 52

### Calculation of Index Value

The measurement was carried out in the same manner as that in Test Example 43, with the exception that 0.5 mL of the antibody solution was fractionated in a test tube, acetone was then added thereto resulting in 50% v/v, the mixture was then left at rest at 25° C. for 24 hours, and nitrogen was then purged for 10 minutes to sublimate the acetone, and the resultant was then freeze-dried at −20° C. Index value 1 (absorption band I, half-value width) obtained by the aforementioned method was 62.0 cm⁻¹.

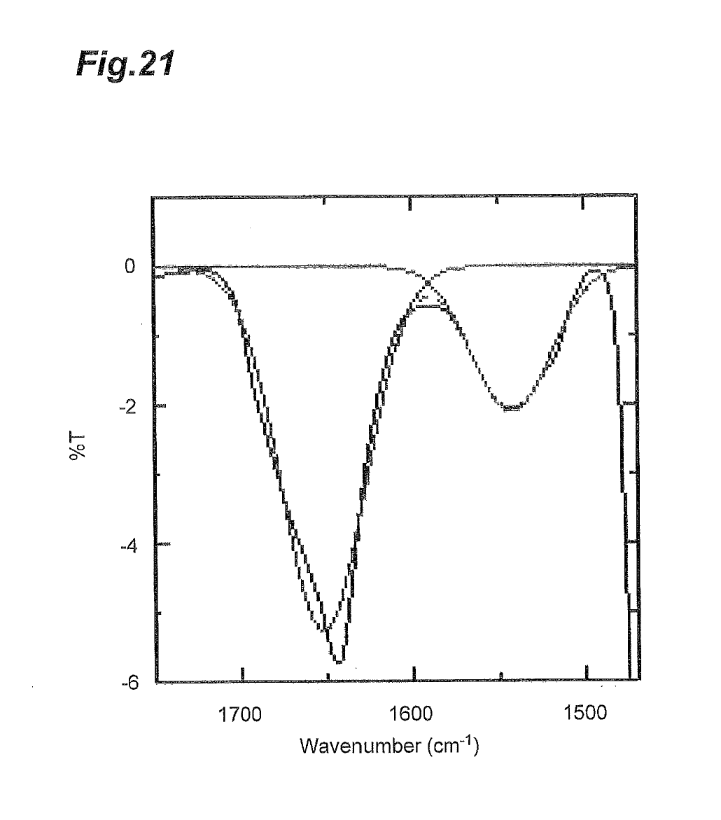

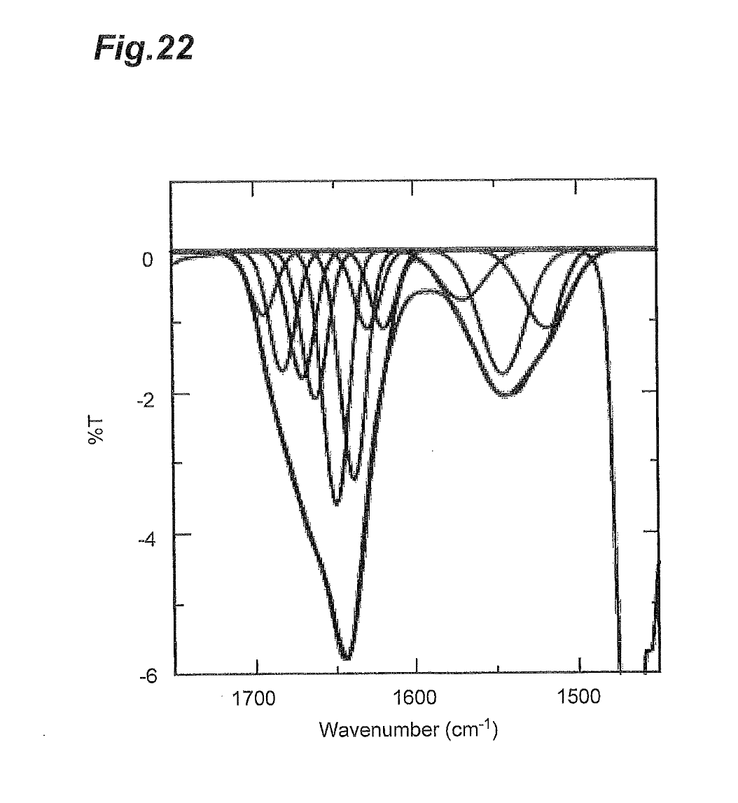

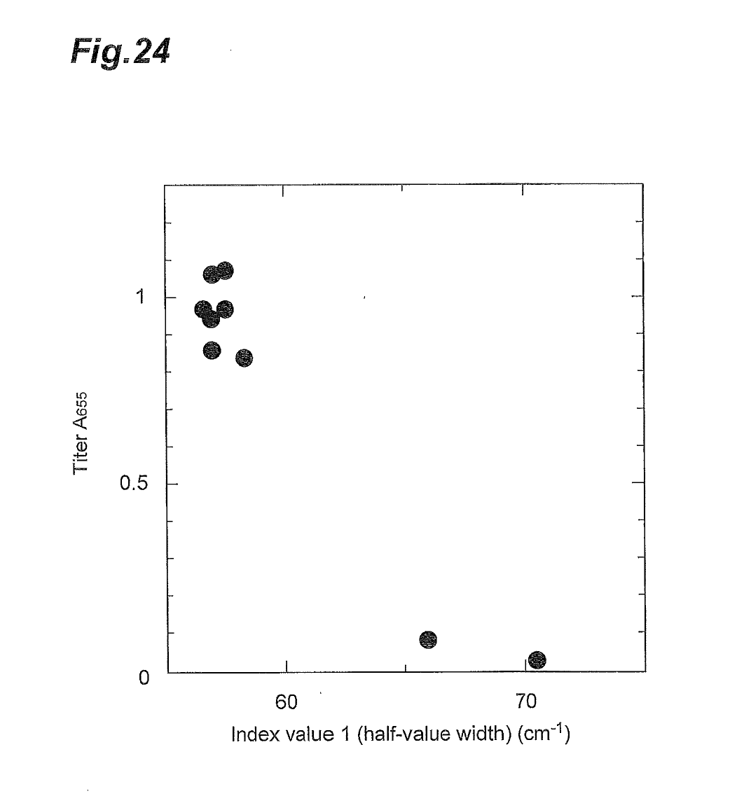

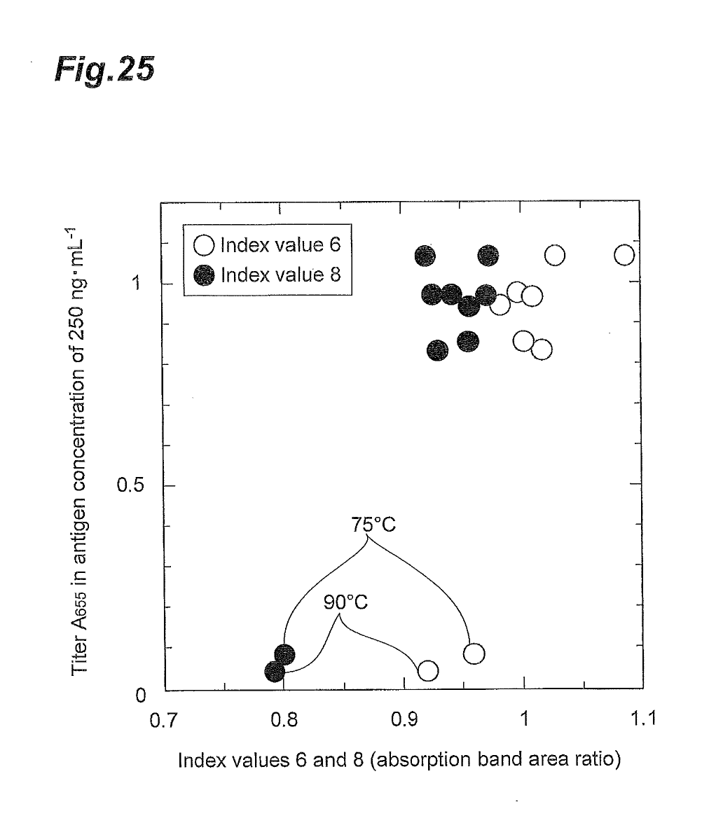

The results of Test Examples 33 to 52 are summarized in the following Table 3. In addition, an example of approximating an infrared absorption band derived from an antibody by a single normal distribution, and an example of subjecting such an infrared absorption band to waveform separation are shown in FIGS. 21 and 22, respectively. FIGS. 24 and 25 each show a graph in which each index value (half-value width or absorption band area ratio) and an antibody titer are plotted.

FIG. 21 shows the FT-IR spectrum of the antibody freeze-dried in Test Example 33, and an example of approximating absorption band I and absorption band II derived from the antibody by a single normal distribution. FIG. 22 shows the FT-IR spectrum of the antibody freeze-dried in Test Example 33, an example of subjecting absorption band I derived from the antibody to waveform separation to obtain eight normal distributions, and an example of subjecting absorption band II to waveform separation to obtain three normal distributions.

FIG. 24 shows a graph in which the titer of an antibody is plotted with respect to index value 1 (absorption band I, half-value width) in each of Test Examples 33 to 42. A linearly approximatable negative correlation is observed.

FIG. 25 shows a graph in which the titer of an antibody is plotted with respect to index value 6 (absorption band I, absorption band area ratio—eight normal distributions) and index value 8 (absorption band II, absorption band area ratio—three normal distributions) in each of Test Examples 33 to 42. A linearly approximatable positive correlation is observed in all of the index values.

TABLE 3

| Test | | | Index | Index | Index | Titer A₆₅₅ in each antigen concentration (ng · mL⁻¹) | | | | | | | |
|---|---|---|---|---|---|---|---|---|---|---|---|---|---|
| Example | Antibody | Treatment | value 1 | value 6 | value 8 | 1000 | 500 | 250 | 125 | 63 | 31 | 16 | 0 |
| 33 | Anti-chicken IgG | −20° C. | 57.0 | 1.09 | 0.97 | 1.06 | 1.05 | 1.06 | 0.91 | 0.69 | 0.44 | 0.31 | 0.05 |
| 34 | Anti-chicken IgG | 4° C. | 57.6 | 1.03 | 0.92 | 1.05 | 1.01 | 1.07 | 0.86 | 0.63 | 0.47 | 0.33 | 0.05 |
| 35 | Anti-chicken IgG | 25° C. | 57.7 | 1.00 | 0.94 | 0.99 | 0.97 | 0.97 | 0.82 | 0.63 | 0.38 | 0.29 | 0.05 |
| 36 | Anti-chicken IgG | 50° C. | 58.4 | 1.02 | 0.93 | 0.90 | 0.89 | 0.84 | 0.49 | 0.39 | 0.24 | 0.18 | 0.05 |
| 37 | Anti-chicken IgG | 75° C. | 66.0 | 0.96 | 0.80 | 0.10 | 0.09 | 0.09 | 0.07 | 0.05 | 0.05 | 0.05 | 0.05 |
| 38 | Anti-chicken IgG | 90° C. | 70.6 | 0.92 | 0.79 | 0.04 | 0.04 | 0.04 | 0.04 | 0.04 | 0.05 | 0.05 | 0.04 |
| 39 | Anti-chicken IgG | 10% Acetone | 56.9 | 0.98 | 0.95 | 1.02 | 0.93 | 0.94 | 0.77 | 0.58 | 0.41 | 0.20 | 0.04 |
| 40 | Anti-chicken IgG | 20% Acetone | 56.7 | 1.01 | 0.97 | 1.02 | 0.99 | 0.97 | 0.75 | 0.65 | 0.36 | 0.29 | 0.04 |
| 41 | Anti-chicken IgG | 40% Acetone | 57.0 | 1.00 | 0.96 | 0.99 | 0.96 | 0.86 | 0.77 | 0.49 | 0.35 | 0.21 | 0.05 |
| 42 | Anti-chicken IgG | 50% Acetone | 57.8 | 1.00 | 0.93 | 1.05 | 1.03 | 0.97 | 0.81 | 0.65 | 0.43 | 0.29 | 0.04 |
| 43 | Anti-chicken egg-white lysozyme | −20° C. | 57.1 | — | — | — | — | — | — | — | — | — | — |
| 44 | Anti-chicken egg-white lysozyme | 4° C. | 57.4 | — | — | — | — | — | — | — | — | — | — |
| 45 | Anti-chicken egg-white lysozyme | 25° C. | 58.6 | — | — | — | — | — | — | — | — | — | — |
| 46 | Anti-chicken egg-white lysozyme | 50° C. | 62.5 | — | — | — | — | — | — | — | — | — | — |
| 47 | Anti-chicken egg-white lysozyme | 75° C. | 72.5 | — | — | — | — | — | — | — | — | — | — |
| 48 | Anti-chicken egg-white lysozyme | 90° C. | 94.0 | — | — | — | — | — | — | — | — | — | — |
| 49 | Anti-chicken egg-white lysozyme | 10% Acetone | 58.4 | — | — | — | — | — | — | — | — | — | — |
| 50 | Anti-chicken egg-white lysozyme | 20% Acetone | 59.0 | — | — | — | — | — | — | — | — | — | — |
| 51 | Anti-chicken egg-white lysozyme | 40% Acetone | 59.9 | — | — | — | — | — | — | — | — | — | — |
| 52 | Anti-chicken egg-white lysozyme | 50% Acetone | 62.0 | — | — | — | — | — | — | — | — | — | — |

As is clear from Table 3 and FIGS. 24 and 25, a linearly approximatable correlation is found between the index value (half-value width or absorption band area ratio) and the antibody titer. Based on this correlation, an antibody, in which half-value width is a predetermined value or less or the absorption band area ratio is a predetermined value or more, can be evaluated to have a sufficient titer.

1. A method for producing a protein, comprising an inspection process, wherein the inspection process comprises:
a step of approximating an infrared absorption band derived from a protein appearing around 1500 to 1600 cm⁻¹ or around 1600 to 1700 cm⁻¹ in an infrared absorption spectrum of the protein, by one or more normal distributions,
a step of calculating an index value indicating a degree of broadening of the infrared absorption band based on the normal distributions, and
a step of comparing the index value with a predetermined threshold to select, as a good-quality product, a protein having a degree of broadening of the infrared absorption band that is smaller than the threshold.

2. The method according to claim 1, wherein the index value is a half-value width of a single normal distribution, when the infrared absorption band is approximated by the single normal distribution.

3. The method according to claim 1, wherein the index value is a value which is obtained by subjecting the infrared absorption band to waveform separation to obtain a plurality of normal distributions, and then dividing a sum of areas of one or more normal distributions around a peak top position of the infrared absorption band by a sum of areas of one or more normal distributions around an end of the infrared absorption band.

4. The method according to claim 1, wherein the index value is a value which is obtained by subjecting the infrared absorption band to waveform separation to obtain two normal distributions each having a peak around a peak top position of the infrared absorption band and having a different half-value width, and then dividing an area of a normal distribution having a smaller half-value width among the two normal distributions by an area of a normal distribution having a larger half-value width.

5. The method according to claim 1, wherein
the index value is a value obtained by subjecting the infrared absorption band to waveform separation to obtain an n number of normal distributions A₁ to Aₙ (wherein n is an integer of 3 or greater), and
when the number n is an even number, by dividing a sum of area(s) of at least one or both of A_{n/2} and A_{n/2+1} by a sum of an area of at least one selected from the group consisting of A₁ to A_{n/2-1} and A_{n/2+2} to Aₙ, or
when the number n is an odd number, by dividing an area of A_{(n-1)/2} by a sum of an area of at least one selected from the group consisting of A₁ to A_{(n-1)/2-1} and A_{(n-1)/2+2} to Aₙ.

6. The method according to claim 1, wherein the protein is an immobilized lipase formed by immobilizing a lipase on a resin carrier.

7. The method according to claim 6, wherein
the index value is a half-value width of a single normal distribution, when an infrared absorption band derived from a lipase appearing around 1600 to 1700 cm⁻¹ is approximated by the single normal distribution, and
in the selection step, an immobilized lipase in which the index value is 70 cm⁻¹ or less is selected as a good-quality product.

8. The method according to claim 6, wherein
the index value is a value obtained by subjecting an infrared absorption band derived from a lipase appearing around 1600 to 1700 cm⁻¹ to waveform separation to obtain two normal distributions A₁ and A₂, and then dividing an area of A₁ by an area of A₂,
with regard to the waveform separation, the infrared absorption band is subjected to the waveform separation to obtain the two normal distributions, A₁ (peak position: 1656 cm⁻¹, half-value width: 47 cm⁻¹) and A₂ (peak position: 1656 cm⁻¹, half-value width: 82 cm⁻¹), so that an absolute value of a difference between an area of the infrared absorption band derived from the lipase and a sum of the areas of the two normal distributions becomes a minimum, and
in the selection step, an immobilized lipase in which the index value is 0.27 or more is selected as a good-quality product.

9. The method according to claim 6, wherein
the index value is a value obtained by subjecting an infrared absorption band derived from a lipase appearing around 1600 to 1700 cm⁻¹ to waveform separation to obtain three normal distributions A₁, A₂ and A₃, and then dividing an area of A₂ by a sum of areas of A₁ and A₃,
with regard to the waveform separation, the infrared absorption band is subjected to the waveform separation to obtain the three normal distributions, A₁ (peak position: 1680 cm⁻¹, half-value width: 50 cm⁻¹), A₂ (peak position: 1656 cm⁻¹, half-value width: 50 cm⁻¹) and A₃ (peak position: 1631 cm⁻¹, half-value width: 50 cm⁻¹), so that an absolute value of a difference between an area of the infrared absorption band derived from the lipase and a sum of the areas of the three normal distributions becomes a minimum, and
in the selection step, an immobilized lipase in which the index value is 0.9 or more is selected as a good-quality product.

10. The method according to claim 6, wherein
the index value is a value obtained by subjecting an infrared absorption band derived from a lipase appearing around 1600 to 1700 cm⁻¹ to waveform separation to obtain five normal distributions A₁, A₂, A₃, A₄ and A₅, and then dividing an area of A₃ by a sum of areas of A₁, A₂, A₄ and A₅,
with regard to the waveform separation, the infrared absorption band is subjected to the waveform separation to obtain the five normal distributions, A₁ (peak position: 1685 cm⁻¹, half-value width: 30 cm⁻¹), A₂ (peak position: 1670 cm⁻¹, half-value width: 30 cm⁻¹), A₃ (peak position: 1656 cm⁻¹, half-value width: 30 cm⁻¹), A₄ (peak position: 1641 cm⁻¹, half-value width: 30 cm⁻¹) and A₅ (peak position: 1626 cm⁻¹, half-value width: 30 cm ⁻¹), so that an absolute value of a difference between an area of the infrared absorption band derived from the lipase and a sum of the areas of the five normal distributions becomes a minimum, and
in the selection step, an immobilized lipase in which the index value is 0.35 or more is selected as a good-quality product.

11. The method according to claim 6, wherein
the index value is a value obtained by subjecting an infrared absorption band derived from a lipase appearing around 1600 to 1700 cm⁻¹ to waveform separation to obtain eight normal distributions A₁, A₂, A₃, A₄, A₅, A₆, A₇ and A₈, and then dividing a sum of areas of A₄ and A₅ by a sum of areas of A₁, A₂, A₃, A₆, A₇ and A₈,
with regard to the waveform separation, the infrared absorption band is subjected to the waveform separation to obtain the eight normal distributions, A₁ (peak position: 1692 cm⁻¹, half-value width: 19 cm⁻¹), A₂ (peak position: 1682 cm⁻¹, half-value width: 19 cm⁻¹), A₃ (peak position: 1670 cm⁻¹, half-value width: 19 cm⁻¹), A₄ (peak position: 1658 cm⁻¹, half-value width: 19 cm⁻¹), A₅ (peak position: 1648 cm⁻¹, half-value width: 19 cm⁻¹), A₆ (peak position: 1638 cm−1, half-value width: 19 cm⁻¹), A₇ (peak position: 1629 cm⁻¹, half-value width: 19 cm⁻¹) and A₈ (peak position: 1619 cm⁻¹, half-value width: 19 cm⁻¹), so that an absolute value of a difference between an area of the infrared absorption band derived from the lipase and a sum of the areas of the eight normal distributions becomes a minimum, and
in the selection step, an immobilized lipase in which the index value is 0.6 or more is selected as a good-quality product.

12. The method according to claim 6, wherein
the index value is a value obtained by subjecting an infrared absorption band derived from a lipase appearing around 1600 to 1700 cm⁻¹ to waveform separation to obtain eight normal distributions A₁, A₂, A₃, A₄, A₅, A₆, A₇ and A₈, and then dividing a sum of areas of A₄ and A₅ by a sum of areas of A₂, A₃ and A₈,
with regard to the waveform separation, the infrared absorption band is subjected to the waveform separation to obtain the eight normal distributions, A₁ (peak position: 1692 cm⁻¹, half-value width: 19 cm⁻¹), A₂ (peak position: 1682 cm⁻¹, half-value width: 19 cm⁻¹), A₃ (peak position: 1670 cm⁻¹, half-value width: 19 cm⁻¹), A₄ (peak position: 1658 cm⁻¹, half-value width: 19 cm⁻¹), A₅ (peak position: 1648 cm⁻¹, half-value width: 19 cm⁻¹), A₆ (peak position: 1638 cm−1, half-value width: 19 cm⁻¹), A₇ (peak position: 1629 cm⁻¹, half-value width: 19 cm⁻¹) and A₈ (peak position: 1619 cm⁻¹, half-value width: 19 cm⁻¹), so that an absolute value of a difference between an area of the infrared absorption band derived from the lipase and a sum of the areas of the eight normal distributions becomes a minimum, and
in the selection step, an immobilized lipase in which the index value is 1.2 or more is selected as a good-quality product.

13. The method according to claim 6, wherein
the index value is a half-value width of a single normal distribution, when an infrared absorption band derived from a lipase appearing around 1500 to 1600 cm⁻¹ is approximated by the single normal distribution, and
in the selection step, an immobilized lipase in which the index value is 44 cm⁻¹ or less is selected as a good-quality product.

14. The method according to claim 6, wherein
the index value is a value obtained by subjecting an infrared absorption band derived from a lipase appearing around 1500 to 1600 cm⁻¹ to waveform separation to obtain three normal distributions B₁, B₂ and B₃, and then dividing an area of B₂ by a sum of areas of B₁ and B₃,
with regard to the waveform separation, the infrared absorption band is subjected to the waveform separation to obtain the three normal distributions, B₁ (peak position: 1570 cm⁻¹, half-value width: 31 cm⁻¹), B₂ (peak position: 1545 cm⁻¹, half-value width: 31 cm⁻¹) and B₃ (peak position: 1518 cm⁻¹, half-value width: 31 cm⁻¹), so that an absolute value of a difference between an area of the infrared absorption band derived from the lipase and a sum of the areas of the three normal distributions becomes a minimum, and
in the selection step, an immobilized lipase in which the index value is 1.2 or more is selected as a good-quality product.

15. The method according to claim 6, wherein the immobilized lipase has a transesterification activity or an ester hydrolysis activity.

16. The method according to claim 6, wherein the lipase is a lipase derived from Burkholderia cepacia or Candida antarctica.

17. The method according to claim 1, wherein the protein is an immobilized peroxidase formed by immobilizing a peroxidase on a silica carrier.

18. The method according to claim 17, wherein
the index value is a half-value width of a single normal distribution, when an infrared absorption band derived from a peroxidase appearing around 1500 to 1600 cm⁻¹ is approximated by the single normal distribution, and
in the selection step, an immobilized peroxidase in which the index value is 75 cm⁻¹ or less is selected as a good-quality product.

19. The method according to claim 17, wherein
the index value is a value obtained by subjecting an infrared absorption band derived from a peroxidase appearing around 1500 to 1600 cm⁻¹ to waveform separation to obtain three normal distributions B₁, B₂ and B₃, and then dividing an area of B₂ by a sum of areas of B₁ and B₃,
with regard to the waveform separation, the infrared absorption band is subjected to the waveform separation to obtain the three normal distributions, B₁ (peak position: 1570 cm⁻¹, half-value width: 31 cm⁻¹), B₂ (peak position: 1545 cm⁻¹, half-value width: 31 cm⁻¹) and B₃ (peak position: 1518 cm⁻¹, half-value width: 31 cm⁻¹), so that an absolute value of a difference between an area of the infrared absorption band derived from the peroxidase and a sum of the areas of the three normal distributions becomes a minimum, and
in the selection step, an immobilized peroxidase in which the index value is 0.45 or more is selected as a good-quality product.

20. The method according to claim 1, wherein the protein is an antibody.

21. The method according to claim 20, wherein
the index value is a half-value width of a single normal distribution, when an infrared absorption band derived from an antibody appearing around 1600 to 1700 cm⁻¹ is approximated by the single normal distribution, and
in the selection step, an antibody in which the index value is 65 cm⁻¹ or less is selected as a good-quality product.

22. The method according to claim 20, wherein
the index value is a value obtained by subjecting an infrared absorption band derived from an antibody appearing around 1600 to 1700 cm⁻¹ to waveform separation to obtain eight normal distributions A₁, A₂, A₃, A₄, A₅, A₆, A₇ and A₈, and then dividing a sum of areas of A₄ and A₅ by a sum of areas of A₂, A₃ and A₈,
with regard to the waveform separation, the infrared absorption band is subjected to the waveform separation to obtain the eight normal distributions, A₁ (peak position: 1692 cm⁻¹, half-value width: 19 cm⁻¹), A₂ (peak position: 1682 cm⁻¹, half-value width: 19 cm⁻¹), A₃ (peak position: 1670 cm⁻¹, half-value width: 19 cm⁻¹), A₄ (peak position: 1658 cm⁻¹, half-value width: 19 cm⁻¹), A₅ (peak position: 1648 cm⁻¹, half-value width: 19 cm⁻¹), A₆ (peak position: 1638 cm−1, half-value width: 19 cm⁻¹), A₇ (peak position: 1629 cm⁻¹, half-value width: 19 cm⁻¹) and A₈ (peak position: 1619 cm⁻¹, half-value width: 19 cm⁻¹), so that an absolute value of a difference between an area of the infrared absorption band derived from the antibody and a sum of the areas of the eight normal distributions becomes a minimum, and
in the selection step, an antibody in which the index value is 0.98 or more is selected as a good-quality product.

23. The method according to claim 20, wherein
the index value is a value obtained by subjecting an infrared absorption band derived from an antibody appearing around 1500 to 1600 cm⁻¹ to waveform separation to obtain three normal distributions B₁, B₂ and B₃, and then dividing an area of B₂ by a sum of areas of B₁ and B₃,
with regard to the waveform separation, the infrared absorption band is subjected to the waveform separation to obtain the three normal distributions, B₁ (peak position: 1570 cm⁻¹, half-value width: 31 cm⁻¹), B₂ (peak position: 1545 cm⁻¹, half-value width: 31 cm⁻¹) and B₃ (peak position: 1518 cm⁻¹, half-value width: 31 cm⁻¹), so that an absolute value of a difference between an area of the infrared absorption band derived from the antibody and a sum of the areas of the three normal distributions becomes a minimum, and
in the selection step, an antibody in which the index value is 0.85 or more is selected as a good-quality product.

24. The method according to claim 1, wherein the infrared absorption spectrum is measured by an attenuated total reflection method.

25. A method for producing a regenerated immobilized lipase, in which a lipase activity is partially or totally regenerated, from an immobilized lipase having a reduced lipase activity, wherein the immobilized lipase is formed by immobilizing a lipase on a resin carrier, and
wherein the method comprises a selection process, the selection process comprising:
a step of approximating an infrared absorption band derived from a lipase appearing around 1500 to 1600 cm⁻¹ or around 1600 to 1700 cm⁻¹ in an infrared absorption spectrum of the immobilized lipase having a reduced lipase activity, by one or more normal distributions,
a step of calculating an index value indicating a degree of broadening of the infrared absorption band based on the normal distributions, and
a step of comparing the index value with a predetermined threshold to select, as an immobilized lipase in which a lipase activity is possibly regenerated, the immobilized lipase having a reduced lipase activity that has a degree of broadening of the infrared absorption band that is smaller than the threshold.

26. A method for evaluating a protein activity, comprising an evaluation process,
wherein the evaluation process comprises:
a step of approximating an infrared absorption band derived from a protein appearing around 1500 to 1600 cm⁻¹ or around 1600 to 1700 cm⁻¹ in an infrared absorption spectrum of the protein, by one or more normal distributions,
a step of calculating an index value indicating a degree of broadening of the infrared absorption band based on the normal distributions, and
a step of comparing the index value with a predetermined threshold to evaluate, as a protein having an activity, a protein having a degree of broadening of the infrared absorption band that is smaller than the threshold.

27. The method for evaluating a protein activity according to claim 26, wherein the index value is a half-value width of a single normal distribution, when the infrared absorption band is approximated by the single normal distribution.

28. The method for evaluating a protein activity according to claim 26, wherein the index value is a value which is obtained by subjecting the infrared absorption band to waveform separation to obtain a plurality of normal distributions, and then dividing a sum of areas of one or more normal distributions around a peak top position of the infrared absorption band by a sum of areas of one or more normal distributions around an end of the infrared absorption band.

29. The method for evaluating a protein activity according to claim 26, wherein the index value is a value which is obtained by subjecting the infrared absorption band to waveform separation to obtain two normal distributions each having a peak around a peak top position of the infrared absorption band and having a different half-value width, and then dividing an area of a normal distribution having a smaller half-value width among the two normal distributions by an area of a normal distribution having a larger half-value width.

30. The method for evaluating a protein activity according to claim 26, wherein
the index value is a value obtained by subjecting the infrared absorption band to waveform separation to obtain an n number of normal distributions A₁ to Aₙ (wherein n is an integer of 3 or greater), and
when the number n is an even number, by dividing a sum of an area(s) of at least one or both of A_{n/2} and A_{n/2+1} by a sum of an area of at least one selected from the group consisting of A₁ to A_{n/2-1} and A_{n/2+2} to Aₙ, or
when the number n is an odd number, by dividing an area of A_{(n-1)/2} by a sum of an area of at least one selected from the group consisting of A₁ to A_{(n-1)/2-1} and A_{(n-1)/2+2} to Aₙ.

31. The method for evaluating a protein activity according to claim 26, wherein the protein is an immobilized lipase formed by immobilizing a lipase on a resin carrier.

32. The method for evaluating a protein activity according to claim 26, wherein the protein is an immobilized peroxidase formed by immobilizing a peroxidase on a silica carrier.

33. The method for evaluating a protein activity according to claim 26, wherein the protein is an antibody.
